SEQUENCE LISTING (418 sequences)

SEQ ID NO: 1 - protein, 61 aa, synthetic construct
  REGION 1..61: note = Synthetic Polypeptide
ATVKFTYQGEEKQVDISKIKKVWRVGQMISFTYDEGGGATGRGAVSEKDAPKELLQMLEKQ

SEQ ID NO: 2 - protein, 62 aa, Sulfolobus solfataricus
ATVKFKYKGEEKQVDISKIKKVWRVGKMISFTYDEGGGKTGRGAVSEKDAPKELLQMLEKQK

SEQ ID NO: 3 - protein, 62 aa, synthetic construct
  REGION 1..62: note = Synthetic Polypeptide
  VARIANT 21..27: note = X can be any naturally occurring amino acid
  VARIANT 21..27: note = Xs cannot sequentially be KKVWRVG
  VARIANT 28..34: note = X can be any naturally occurring amino acid
  VARIANT 28..34: note = Xs cannot sequentially be QMISFTY
  VARIANT 40..46: note = X can be any naturally occurring amino acid
  VARIANT 40..46: note = Xs cannot sequentially be ATGRGAV
ATVKFTYQGEEKQVDISKIKXXXXXXXXXXXXXXDEGGGXXXXXXXSEKDAPKELLQMLEKQ

SEQ ID NO: 4 - protein, 62 aa, synthetic construct
  REGION 1..62: note = Synthetic Polypeptide
  VARIANT 22: note = X can be any naturally occurring amino acid
  VARIANT 22..45: note = Xs cannot simultaneously be K, W, V, M, S, T, T, R, and A, sequentially
  VARIANT 24: note = X can be any naturally occurring amino acid
  VARIANT 26: note = X can be any naturally occurring amino acid
  VARIANT 29: note = X can be any naturally occurring amino acid
  VARIANT 31: note = X can be any naturally occurring amino acid
  VARIANT 33: note = X can be any naturally occurring amino acid
  VARIANT 41: note = X can be any naturally occurring amino acid
  VARIANT 43: note = X can be any naturally occurring amino acid
  VARIANT 45: note = X can be any naturally occurring amino acid
ATVKFTYQGEEKQVDISKIKKXVXRXGQXIXFXYDEGGGAXGXGXVSEKDAPKELLQMLEKQ

SEQ ID NO: 5 - protein, 61 aa, synthetic construct
  REGION 1..61: note = Synthetic Polypeptide
ATVKFTYQGEEKQVDISKIKWVIRWGQHIAFKYDEGGGAAGYGWVSEKDAPKELLQMLEKQ

SEQ ID NO: 6 - protein, 61 aa, synthetic construct
  REGION 1..61: note = Synthetic Polypeptide
ATVKFTYQGEEKQVDISKIKWVNRWGQRIYFKYDEGGGAAGYGWVSEKDAPKELLQMLEKQ

SEQ ID NO: 7 - protein, 61 aa, synthetic construct
  REGION 1..61: note = Synthetic Polypeptide
ATVKYTYRGEEKRVDISKIKWVNRWGQHLAFKYDKGGGAAGYGWVSEKDAPKELLQMLEKR

SEQ ID NO: 8 - protein, 61 aa, synthetic construct
  REGION 1..61: note = Synthetic Polypeptide
ATVKSTYRGEEKQVDISKIKWVIRWGQHLAFKYDEGGGAAGYGWVSEKDAPKELLQMLEKQ

SEQ ID NO: 9 - protein, 61 aa, synthetic construct
  REGION 1..61: note = Synthetic Polypeptide
ATVKFTYRGEEKQVDISKIKWVNRWGQHLAFKYDVGGGAAGYGWMSEKDAPKELLQMLEKR

SEQ ID NO: 10 - protein, 61 aa, synthetic construct
  REGION 1..61: note = Synthetic Polypeptide
ATVKFTYQGEEKQVDISKIKWVIRLGRTIMFKYDEGGGANGYGKVSEKDAPKELLQMLEKQ

SEQ ID NO: 11 - protein, 61 aa, synthetic construct
  REGION 1..61: note = Synthetic Polypeptide
ATVKFTYQGEEKQVDISKIKWVVRLGQVIMFKYDEGGGANGYGKVSEKDAPKELLQMLEKQ

SEQ ID NO: 12 - protein, 61 aa, synthetic construct
  REGION 1..61: note = Synthetic Polypeptide
ATVKFTYRGEEKQVDISKIKWVVRLGQVIMFKYGEGGGSNGYGRVSEKDAPKELRQMLEKR

SEQ ID NO: 13 - protein, 60 aa, synthetic construct
  REGION 1..60: note = Synthetic Polypeptide
ATVKFTYRGEEKQVDISKIKWVVRLGQVIMFKYDEGGGASGYGRVSEKDAPKELLQMLEK

SEQ ID NO: 14 - protein, 73 aa, synthetic construct
  REGION 1..73: note = Synthetic Polypeptide
ATVKFTYRGEEKQVGVSRVKSVHRIGQWIKFWYDEGSGAYGRGYVSEKDAPEELLQMLEKRGSEQKLISEEDL

SEQ ID NO: 15 - protein, 7 aa, synthetic construct
  REGION 1..7: note = Synthetic Polypeptide
ATVKFTY

SEQ ID NO: 16 - protein, 18 aa, synthetic construct
  REGION 1..18: note = Synthetic Polypeptide
GVSRVKSVHRIGQWIKFW

SEQ ID NO: 17 - protein, 5 aa, synthetic construct
  REGION 1..5: note = Synthetic Polypeptide
  VARIANT 3: note = X can be any naturally occurring amino acid
LPXTG

SEQ ID NO: 18 - protein, 5 aa, synthetic construct
  REGION 1..5: note = Synthetic Polypeptide
  VARIANT 3: note = X can be any naturally occurring amino acid
LRXTG

SEQ ID NO: 19 - protein, 5 aa, synthetic construct
  REGION 1..5: note = Synthetic Polypeptide
GGGGS

SEQ ID NO: 20 - protein, 10 aa, synthetic construct
  REGION 1..10: note = Synthetic Polypeptide
GGGGSGGGGS

SEQ ID NO: 21 - protein, 15 aa, synthetic construct
  REGION 1..15: note = Synthetic Polypeptide
GGGGSGGGGSGGGGS

SEQ ID NO: 22 - protein, 20 aa, synthetic construct
  REGION 1..20: note = Synthetic Polypeptide
GGGGSGGGGSGGGGSGGGGS

SEQ ID NO: 23 - protein, 25 aa, synthetic construct
  REGION 1..25: note = Synthetic Polypeptide
GGGGSGGGGSGGGGSGGGGSGGGGS

SEQ ID NO: 24 - protein, 30 aa, synthetic construct
  REGION 1..30: note = Synthetic Polypeptide
GGGGSGGGGSGGGGSGGGGSGGGGSGGGGS

SEQ ID NO: 25 - protein, 72 aa, synthetic construct
  REGION 1..72: note = Synthetic Polypeptide
ATVKFTYQGEEKQVDISKIKHVRRWGRWIWFIYEEGGGAKGRGGVSEKDAPKELLQMLEKQGSEQKLISEED

SEQ ID NO: 26 - protein, 72 aa, synthetic construct
  REGION 1..72: note = Synthetic Polypeptide
ATGKFTYQGEKKQGDISKIKHGRRWGRGIWFIYEEGGGAKGRGGVSEKDAPKELLQMLEKQGSEQKLISEED

SEQ ID NO: 27 - protein, 72 aa, synthetic construct
  REGION 1..72: note = Synthetic Polypeptide
ITVKFTYQGEEKQVDISKIEHVRRWGQWIWFTYDEGGGAKGRGGVSEKGAPKELLQMLGKRGSEQKLISEED

SEQ ID NO: 28 - protein, 72 aa, synthetic construct
  REGION 1..72: note = Synthetic Polypeptide
ATVKFTYQGEEKQVDISKIKHVRRWGQWIWFIYDKGGGAKGRGGVSEKDAPKELLQMLEKQGSEQKLISEED

SEQ ID NO: 29 - protein, 69 aa, synthetic construct
  REGION 1..69: note = Synthetic Polypeptide
ATVKFTYQGEEKQVDISKIKHVRRWGQWIWFIYDEGGGAKGKGSVSEKDAPKELLQMLEKAGIRTKAYF

SEQ ID NO: 30 - protein, 72 aa, synthetic construct
  REGION 1..72: note = Synthetic Polypeptide
ATVEFTYQGEEKQVDISKIKHVRRWGQWIWFIYDEGGGAKGRGGVSERDAPKELLQLLEKRGSEQKLISEED

SEQ ID NO: 31 - protein, 69 aa, synthetic construct
  REGION 1..69: note = Synthetic Polypeptide
ATVKFTYQGEEKQVDISKIKYVRRWGQAIIFRYDEGGGAEGKGSVSEKGAPKELLQMLEKARIRTKAYF

SEQ ID NO: 32 - protein, 72 aa, synthetic construct
  REGION 1..72: note = Synthetic Polypeptide
ATVKFTYRGEEKQVGISRIKSVHRIGQWIKFWYDEGSGAYGRGYVSEKDAPKELLQMLEKRGSEQKLISEED

SEQ ID NO: 33 - protein, 72 aa, synthetic construct
  REGION 1..72: note = Synthetic Polypeptide
AIVKFTYQGEEKQVDISKIKYVRRWGQYIWFGYDEGGGAHGRGRVSGKDAPKELLQMLEKQGSEQKLISEED

SEQ ID NO: 34 - protein, 69 aa, synthetic construct
  REGION 1..69: note = Synthetic Polypeptide
ATVKFTYRGEEKQVDISKIKSVSRWGQAIIFRYDGGGGARGKGSVSEKDAPKELLQMLEEARIRTKAYF

SEQ ID NO: 35 - protein, 68 aa, synthetic construct
  REGION 1..68: note = Synthetic Polypeptide
ATVKFTYRGEEKQVGISRIKSVHRIGQWIKFWYDEGSGAYGRGYVSEKDAPKELLQMLEKRGSEQKLF

SEQ ID NO: 36 - protein, 60 aa, synthetic construct
  REGION 1..60: note = Synthetic Polypeptide
ATVKFTYRGKEKQVGISRIKSVHRIGQWIRFWYDEGSGAYGRGYVSEKDAPKELLQMLEK

SEQ ID NO: 37 - protein, 72 aa, synthetic construct
  REGION 1..72: note = Synthetic Polypeptide
ATVKFTYRGEEKQVGISRIKSVHRIGQWIKFWYDEGSGAYGRGYVSGKDAPKELLQMLEKRGSEQKLISEED

SEQ ID NO: 38 - protein, 72 aa, synthetic construct
  REGION 1..72: note = Synthetic Polypeptide
ATVKFTYRGKEKQVGISRIKSVHRIGQWIKFWYDEGSGAYGRGYVSGKDAPKELLQMLEKRGSEQKLISEED

SEQ ID NO: 39 - protein, 72 aa, synthetic construct
  REGION 1..72: note = Synthetic Polypeptide
ATVKFTYRGEEKQVGINRIKSVHRIGQWIKFWYDEGSGAYGRGYVSGKDAPKELLRMLEKRGSEQKLISEED

SEQ ID NO: 40 - protein, 72 aa, synthetic construct
  REGION 1..72: note = Synthetic Polypeptide
ATVKFTYRGEEKQVGISRIKSVHRIGRWIKFWYDEGSGAYGRGYVSGKDAPKELLQMLEKRGSEQKLISEED

SEQ ID NO: 41 - protein, 72 aa, synthetic construct
  REGION 1..72: note = Synthetic Polypeptide
ATVKFTYRGEEKQVGISRIKSVRRIGQWIKFWYDEGSGAYGRGYVSGKDAPKELLQMLEKRGSEQKLISEED

SEQ ID NO: 42 - protein, 72 aa, synthetic construct
  REGION 1..72: note = Synthetic Polypeptide
ATVKFTYRGEEKRVGISRIKSVRRIGQWIKFWYDEGSGAYGRGYVSEKDAPKELLQMLGKRGSEQKLISEED

SEQ ID NO: 43 - protein, 72 aa, synthetic construct
  REGION 1..72: note = Synthetic Polypeptide
ATVKFTYRGEEKQVGISRIKSVRRIGQWIKFWYDEGSGAYGRGYVSEKDAPKELLQMLGKRGSEQKLISEED

SEQ ID NO: 44 - protein, 72 aa, synthetic construct
  REGION 1..72: note = Synthetic Polypeptide
ATVRFTYRGEEKQVGISRIKSVRRIGQWIKFWYDEGSGAYGRGYVSEKDAPKELLQMLGKRGSEQKLISEED

SEQ ID NO: 45 - protein, 72 aa, synthetic construct
  REGION 1..72: note = Synthetic Polypeptide
ATVKFTYRGEEKQVGISRIKSVHRIGQWIKFWYDEGSGAYGRGYVSEKGAPKELLQMLGKRGSEQKLISEED

SEQ ID NO: 46 - protein, 72 aa, synthetic construct
  REGION 1..72: note = Synthetic Polypeptide
ATVKFTYRGEEKQVGISRIRSVHRIGQWIKFWYDEGSGACGRGYVSEKGAPKELLQMLGKRGSEQKLISEED

SEQ ID NO: 47 - protein, 72 aa, synthetic construct
  REGION 1..72: note = Synthetic Polypeptide
ATVKFTYRGEEKQVGISRIKSVHRIGQWIKFWYDEGSGAYGRGYVSEKDAPKELLQMLGKRGSEQKLISEED

SEQ ID NO: 48 - protein, 72 aa, synthetic construct
  REGION 1..72: note = Synthetic Polypeptide
ATVRFTYRGEEKQVGISRIKSVHRIGQWIKFWYDEGSGTYGRGYVSEKDAPRELLQMLGKRGSEQKLISEED

SEQ ID NO: 49 - protein, 72 aa, synthetic construct
  REGION 1..72: note = Synthetic Polypeptide
ATVKFTYRGEEKQVGISRIKSVRRIGQWIKFWYDEGSGAYGRGYVSEKDAPKELLQMLEKRGSEQKLISEED

SEQ ID NO: 50 - protein, 72 aa, synthetic construct
  REGION 1..72: note = Synthetic Polypeptide
ATVKFTYRGGEKQVGISRIKSVRRIGQWIKFWYDEGSGAYGRGYVSEKDAPKELLQMLEKRGSEQKLISEED

SEQ ID NO: 51 - protein, 72 aa, synthetic construct
  REGION 1..72: note = Synthetic Polypeptide
ATVKFTYRGKEKRVGVSRIKSVRRIGQWIKFWYDEGSGAYGRGYVSEKDAPKELLQMLEKRGSEQKLISEED

SEQ ID NO: 52 - protein, 72 aa, synthetic construct
  REGION 1..72: note = Synthetic Polypeptide
ATVKFTYRGEEKRVGISRIKSVRRIGQWIKFWYDEGSGAYGRGYVSEKDAPKELLQMLEKRGSEQKLISEED

SEQ ID NO: 53 - protein, 72 aa, synthetic construct
  REGION 1..72: note = Synthetic Polypeptide
ATVKFTYRGEEKQVGISRIKSVRRIGQWVKFWYDEGSGAYGRGYVSEKDAPKELLQMLEKRGSEQKLISEED

SEQ ID NO: 54 - protein, 72 aa, synthetic construct
  REGION 1..72: note = Synthetic Polypeptide
ATVKFTYRGEEKQVGISRIKSVRRIGQWVKFWYGEGSGAYGRGYVSEKDAPKELLQMLEKRGSEQKLISEED

SEQ ID NO: 55 - protein, 72 aa, synthetic construct
  REGION 1..72: note = Synthetic Polypeptide
ATVKFTYRGEEKQVGISRIRSVRRIGQWIKFWYDEGSGAYGRGYVSEKDAPKELLQMLEKRGSEQKLISEED

SEQ ID NO: 56 - protein, 72 aa, synthetic construct
  REGION 1..72: note = Synthetic Polypeptide
ATVKFTYRGEEKQVGISRIKSVRRIGQWIKFWYDEGRGAYGRGYVSEKDAPKELLQMLEKRGSEQKLISEED

SEQ ID NO: 57 - protein, 72 aa, synthetic construct
  REGION 1..72: note = Synthetic Polypeptide
ATVKFTYRGEEKQVGISRIKSVRRIGQWIKFWYDEGSGAYGRGYVSEKDAPEELLQMLEKRGSEQKLISEED

SEQ ID NO: 58 - protein, 72 aa, synthetic construct
  REGION 1..72: note = Synthetic Polypeptide
ATVKFTYRGEEKQVGISRIKSVHRIGQWIKFWYDEGSGAYGRGYVSKKDAPKELLQMLEKRGSEQKLISEED

SEQ ID NO: 59 - protein, 72 aa, synthetic construct
  REGION 1..72: note = Synthetic Polypeptide
ATVKFTYRGEEKQVGVSRIKSVHRIGRWIKFWYDEGSGAYGRGYVSEKDAPKELLQMLEKRGSEQKLISEED

SEQ ID NO: 60 - protein, 72 aa, synthetic construct
  REGION 1..72: note = Synthetic Polypeptide
ATVKFTYRGEEKQVGISRIKSVHRIGRWIKFWYDEGSGAYGRGYVSEKDAPKELLQMLEKRGSEQKLISEED

SEQ ID NO: 61 - protein, 72 aa, synthetic construct
  REGION 1..72: note = Synthetic Polypeptide
ATVKFTYRGEEKQVGISRIKSVHRVGRWIKFWYDEGSGAYGRGYVSEKDAPKELLQMLEKRGSEQKLISEED

SEQ ID NO: 62 - protein, 72 aa, synthetic construct
  REGION 1..72: note = Synthetic Polypeptide
ATVKFTYRGEEKQVGIGRIKSVHRIGRWIKFWYDEGSGAYGRGYVSEKDAPKELLQMLEKRGSEQKLISEED

SEQ ID NO: 63 - protein, 72 aa, synthetic construct
  REGION 1..72: note = Synthetic Polypeptide
ATVKFTYRGEEKQVGISRIKSVHRIGRWIKFWYDEGSGAYGRGYVNEKDAPKELLQMLEKRGSEQKLISEED

SEQ ID NO: 64 - protein, 72 aa, synthetic construct
  REGION 1..72: note = Synthetic Polypeptide
ATVKFTYRGEEKQVGISRIKFVHRIGRWIKFWYDEGSGAYGRGYVSEKDAPKELLQMLEKRGSEQKLISEED

SEQ ID NO: 65 - protein, 72 aa, synthetic construct
  REGION 1..72: note = Synthetic Polypeptide
ATVKFTYRGEEKQVGISRIKSVHRIGQWIKFWYDEGSGAYGRGYVSEKNAPKELLQMLEKRGSEQKLISEED

SEQ ID NO: 66 - protein, 72 aa, synthetic construct
  REGION 1..72: note = Synthetic Polypeptide
ATVKFTYRGEEKQVGISRIKSVHRIGQWIKFWYDEGSGAYGRGYVSEKGAPKELLQMLEKRGSEQKLISEED

SEQ ID NO: 67 - protein, 72 aa, synthetic construct
  REGION 1..72: note = Synthetic Polypeptide
ATVKFTYRGEGKQVGISRIKSVHRIGQWIKFWYDEGSGAYGRGYVSEKDAPKELLQMLEKRGSEQKLISEED

SEQ ID NO: 68 - protein, 72 aa, synthetic construct
  REGION 1..72: note = Synthetic Polypeptide
ATVKFTYRGEGKQVGISRIKSVHRIGQWIKFWYDEGSGAYGRGYVSEKDAPKELLQMLKKRGSEQKLISEED

SEQ ID NO: 69 - protein, 72 aa, synthetic construct
  REGION 1..72: note = Synthetic Polypeptide
ATVKFTYRGERKQVGISRIKSVHRIGQWIKFWYDEGSGAYGRGYVSEKDAPKELLQMLEKRGSEQKLISEED

SEQ ID NO: 70 - protein, 72 aa, synthetic construct
  REGION 1..72: note = Synthetic Polypeptide
ATVKFTYRGEEKQVGISRIKSVHRVGQWIKFWYDEGSGAYGRGYVSEKDAPKELLQMLEKRGSEQKLISEED

SEQ ID NO: 71 - protein, 72 aa, synthetic construct
  REGION 1..72: note = Synthetic Polypeptide
ATVKFTYRGEERQVGISRIKSVHRIGQWIKFWYDEGSGAYGRGYVSEKDAPKELLQMLEKRGSEQKLISEED

SEQ ID NO: 72 - protein, 72 aa, synthetic construct
  REGION 1..72: note = Synthetic Polypeptide
ATVKFTYRGEEKQVGISRIKSVHRIGQWIKFWYDEGSGAYGRGYVGEKDAPKELLQMLEKRGSEQKLISEED

SEQ ID NO: 73 - protein, 72 aa, synthetic construct
  REGION 1..72: note = Synthetic Polypeptide
ATVKFTYRGEEKRVGISRIKSVHRIGQWIKFWYDEGSGAYGRGYVSEKDAPKELLQMLEKRGSEQKLISEED

SEQ ID NO: 74 - protein, 72 aa, synthetic construct
  REGION 1..72: note = Synthetic Polypeptide
ATVKFTYRGEEKQVGISRIKSVHRIGQWIKFWYDEGSGAYGRGYVSEKDAPKELLRMLEKRGSEQKLISEED

SEQ ID NO: 75 - protein, 72 aa, synthetic construct
  REGION 1..72: note = Synthetic Polypeptide
ATVKFTYRGEEKQVGVSRIKSVHRIGQWIKFWYDGGSGAYGRGYVSEKDAPKELLQMLEKRGSEQKLISEED

SEQ ID NO: 76 - protein, 72 aa, synthetic construct
  REGION 1..72: note = Synthetic Polypeptide
ATVKFTYRGEEKQVGISRIKSVHRIGQWIKFWYDGGSGAYGRGYVSEKDAPKELLQMLEKRGSEQKLISEED

SEQ ID NO: 77 - protein, 72 aa, synthetic construct
  REGION 1..72: note = Synthetic Polypeptide
ATVKFTYRGEEKQVGVSRVKSVHRIGQWIKFWYDEGSGAYGRGYVSEKDAPEELLQMLEKRGSEQKLISEED

SEQ ID NO: 78 - protein, 72 aa, synthetic construct
  REGION 1..72: note = Synthetic Polypeptide
ATVKFTYRGEEKQVGISRIKSVHRIGQWIKFWYDEGSGAHGRGYVSEKDAPKELLQMLEKRGSEQKLISEED

SEQ ID NO: 79 - protein, 72 aa, synthetic construct
  REGION 1..72: note = Synthetic Polypeptide
ATVKFTYRGEEKQVGISRIKSVHRIGQWIKFRYDEGSGAYGRGYVSEKDAPKELLQMLEKRGSEQKLISEED

SEQ ID NO: 80 - protein, 72 aa, synthetic construct
  REGION 1..72: note = Synthetic Polypeptide
VTVEFTYRGEEKQVDISKIKHVRRWGRWIWFIYEEGGGAKGRGGVSEKDAPKELLQMLEKRGSEQKLISEED

SEQ ID NO: 81 - protein, 72 aa, synthetic construct
  REGION 1..72: note = Synthetic Polypeptide
ATVKFTYQGEEKQVDISKIKHVRRWGRWIWFIYEEGGGAKGRGGVSEKDAPKELLQMLEKRGSEQKLISEED

SEQ ID NO: 82 - protein, 72 aa, synthetic construct
  REGION 1..72: note = Synthetic Polypeptide
ATVRFTYQGEEKQVDISKIKHVRRWGRWIWFIYEEGGGAKGRGGVSEKDAPKELLQMLEKRGSEQKLISEED

SEQ ID NO: 83 - protein, 72 aa, synthetic construct
  REGION 1..72: note = Synthetic Polypeptide
ATVKFTYQGEEKQVDISKIKHVRRWGRWIWFIYEEGGGGKGRGGVSEKDAPKELLQMLEKRGSEQKLISEED

SEQ ID NO: 84 - protein, 72 aa, synthetic construct
  REGION 1..72: note = Synthetic Polypeptide
ATVKFTHQGEEKQVDISKIKHVRRWGRWIWFIYEEGGGAKGRGGVSEKDAPKELLQMLEKRGSEQKLISEED

SEQ ID NO: 85 - protein, 72 aa, synthetic construct
  REGION 1..72: note = Synthetic Polypeptide
AIVKFTYQGEEKQVDISKIKHVRRWGRWIWFIYEEGGGAKGRGGVSEKDAPKELLQMLEKRGSEQKLISEED

SEQ ID NO: 86 - protein, 72 aa, synthetic construct
  REGION 1..72: note = Synthetic Polypeptide
AAVKFTYQGEEKQVDISKIKHVRRWGRWIWFIYEEGGGAKGRGGVSEKDAPKELLQMLEKQGSEQKLISEED

SEQ ID NO: 87 - protein, 72 aa, synthetic construct
  REGION 1..72: note = Synthetic Polypeptide
ATVKFTYQGEEKQVDISKIKHVRRWGRWIWFIYEAGGGAKGRGGVSEKDAPKELLQMLEKQGSEQKLISEED

SEQ ID NO: 88 - protein, 72 aa, synthetic construct
  REGION 1..72: note = Synthetic Polypeptide
ATVKFTYQGEEKQVDISKIKHVRRWGRWIWFIYEGGGGAKGRGGVSEKDAPKELLQMLERRGSEQKLISEED

SEQ ID NO: 89 - protein, 72 aa, synthetic construct
  REGION 1..72: note = Synthetic Polypeptide
ATVKFTYQGEEKQVDISKIKHVRRWGRWIWFIYEGGGGAKGRGGVSEKDAPKELLQMLEKQGSEQKLISEED

SEQ ID NO: 90 - protein, 69 aa, synthetic construct
  REGION 1..69: note = Synthetic Polypeptide
ATVKFTYRGEEKQVDISKIKHVRRWGQWIWFIYDEGGGAKGKGSVSEKDAPKELLQMLEKAGIRTKAYF

SEQ ID NO: 91 - protein, 69 aa, synthetic construct
  REGION 1..69: note = Synthetic Polypeptide
ATVKFTYQGEEKQVDVSKIKHVRRWGQWIWFIYDEGGGAKGKGSVSEKDAPKELLQMLGKAGIRTKAYF

SEQ ID NO: 92 - protein, 69 aa, synthetic construct
  REGION 1..69: note = Synthetic Polypeptide
ATVKFTYQGEEKQVDISKIKHVRRWGRWIWFIYDEGGGAKGKGSVSEKDAPKELLQMLEKAGIRTKAYF

SEQ ID NO: 93 - protein, 69 aa, synthetic construct
  REGION 1..69: note = Synthetic Polypeptide
ATVKFTYQGEEKQVDISKIKHVRRWGRWIWFIYEEGGGAKGKGSVSEKDAPKELLQMLEKAGIRTKAYF

SEQ ID NO: 94 - protein, 69 aa, synthetic construct
  REGION 1..69: note = Synthetic Polypeptide
ATVKFTYQGEEKQVDISKIKHVRRWGQRIWFIYGEGGGAKGRGSVSEKDAPKELLQMLEKAGIRTKAYF

SEQ ID NO: 95 - protein, 72 aa, synthetic construct
  REGION 1..72: note = Synthetic Polypeptide
ATVKFTYQGEEKQVDISKIKHVRRWGRWIWFIYEEGGAKGRGGVSEKDAPKELLQMLEKQGSEQKLISEEDL

SEQ ID NO: 96 - protein, 72 aa, synthetic construct
  REGION 1..72: note = Synthetic Polypeptide
ATVKFTYQGEEKQVDISKIKHVRRWGRWIWFTYEEGGGAKGRGGVSEKDAPKELLQMLEKQGSEQKLILKRT

SEQ ID NO: 97 - protein, 72 aa, synthetic construct
  REGION 1..72: note = Synthetic Polypeptide
ATVKFTYQGEEKQVDISKIKHVRRWGRWIWFTYEEGGGAKGRGGVSEKDAPKELLQMLEKPGSEQKLISEED

SEQ ID NO: 98 - protein, 72 aa, synthetic construct
  REGION 1..72: note = Synthetic Polypeptide
ATVKFTYQGEEKQVDISKIKHVRRWGRWIWFTYEEGGGARGRGGVSEKDAPKELLQMLEKQGSEQKLISEED

SEQ ID NO: 99 - protein, 72 aa, synthetic construct
  REGION 1..72: note = Synthetic Polypeptide
ATVKFTYQGEEKQVDVSKIKHVRRWGRWIWFTYEEGGGAKGRGGVSEKDAPKELLQMLEKQGSEQKLISEED

SEQ ID NO: 100 - protein, 72 aa, synthetic construct
  REGION 1..72: note = Synthetic Polypeptide
ATVKFTYQGEEKQVDISKIKHVRRWGRWIWFTYEEGGGAKGRGGVSEKDAPRELLQMLEKRGSEQKLISEED

SEQ ID NO: 101 - protein, 72 aa, synthetic construct
  REGION 1..72: note = Synthetic Polypeptide
ATVKFTYRGEEKQVDISEIKHVRRWGRWIWFTYEEGGGARGRGGVSEKDAPKELLQMLEKRGSEQKLISEED

SEQ ID NO: 102 - protein, 72 aa, synthetic construct
  REGION 1..72: note = Synthetic Polypeptide
ATVKFTYQGEEKQVDISKIRHVRRWGRRIWFTYEEGGGAKGRGGVGEKDAPKELLQMLEKQGSEQKLISEED

SEQ ID NO: 103 - protein, 72 aa, synthetic construct
  REGION 1..72: note = Synthetic Polypeptide
ATVKFTYQGEEKQVDISKIKHVRRWGRRIWFIYEEGGGAKGRGGVSEKDAPKELLQMLEKQGSEQKLISEKD

SEQ ID NO: 104 - protein, 72 aa, synthetic construct
  REGION 1..72: note = Synthetic Polypeptide
ATVKFTYQGEEKQVDISKIKHVRRWGRRIWFIYEEGGGAKGRGGVSEKDAPKELLQMLEKQGSEQKLISEED

SEQ ID NO: 105 - protein, 72 aa, synthetic construct
  REGION 1..72: note = Synthetic Polypeptide
ATVKFTYQGEEKQVDISKIKHVRRWGRWIWFIYEEGGGAKGRGGVGEKDAPKELLQMLEKQGSEQKLISEED

SEQ ID NO: 106 - protein, 72 aa, synthetic construct
  REGION 1..72: note = Synthetic Polypeptide
ATVKFTYQGEGKQVDISKIKHVRRWGRWIWFIYEEGGGAKGRGGVSEKDAPKELLQMLEKQGSEQKLISEED

SEQ ID NO: 107 - protein, 72 aa, synthetic construct
  REGION 1..72: note = Synthetic Polypeptide
ATVKFTYQGEEKQVDISKIKRVRRWGRWIWFIYEEGGGAKGRGGVSEKDAPKELLQMLEKQGSEQKLISEED

SEQ ID NO: 108 - protein, 72 aa, synthetic construct
  REGION 1..72: note = Synthetic Polypeptide
ITVKFTYQGEEKQVDISKIEHVRRWGRWIWFTYDEGGGAKGRGGVSEKGAPKELLQMLGKRGSEQKLISEED

SEQ ID NO: 109 - protein, 72 aa, synthetic construct
  REGION 1..72: note = Synthetic Polypeptide
ITVKFTYRGEEKQVDISKIEHVRRWGQWIWFTYDEGGGAKGRGGVSEKGAPRELLQMLGKRGSEQKLISEED

SEQ ID NO: 110 - protein, 72 aa, synthetic construct
  REGION 1..72: note = Synthetic Polypeptide
ATVKFTYQGEEKQVDISKIKHVRRWGRWIWFTYDEGGGAKGRGGVSEKGAPKELLQMLGKRGSEQKLISEED

SEQ ID NO: 111 - protein, 72 aa, synthetic construct
  REGION 1..72: note = Synthetic Polypeptide
ATVKFTYQGEEKQVDISKIKHVRRWGRWIWFTYEEGGGAKGRGGVSEKDAPKELLQMLGKRGSEQKLISEED

SEQ ID NO: 112 - protein, 72 aa, synthetic construct
  REGION 1..72: note = Synthetic Polypeptide
ATVKFTYQGEEKRVDISKIKHVRRWGRWIWFIYEEGGGAKGRGGVSEKDAPKELLQMLGKRGSEQKLISEED

SEQ ID NO: 113 - protein, 72 aa, synthetic construct
  REGION 1..72: note = Synthetic Polypeptide
ATVKFTYQGEEKQVDISKIKHVRRWGRWIWFIYEEGGGAKGRGGVSEKDAPKELLQMLGKQGSEQKLISEED

SEQ ID NO: 114 - protein, 72 aa, synthetic construct
  REGION 1..72: note = Synthetic Polypeptide
ATVKFTYRGEEKQVDISKIKHVRRWGRWIWFIYEEGGGAKGRGGVSEKDAPKELLQMLGKQRSEQKLISEED

SEQ ID NO: 115 - protein, 72 aa, synthetic construct
  REGION 1..72: note = Synthetic Polypeptide
ATVKFTYHGEEKQVDISKIKHVRRWGRWIWFIYEEGGGAKGRGGVSEKDAPKELLQMLEKQGSEQKLISEED

SEQ ID NO: 116 - protein, 72 aa, synthetic construct
  REGION 1..72: note = Synthetic Polypeptide
ATVKFTYRGEEKQVDISKIKHVRRWGQWIWFIYDKGGGAKGRGGVSEKDAPKELLQMLEKQGSEQKLISEED

SEQ ID NO: 117 - protein, 72 aa, synthetic construct
  REGION 1..72: note = Synthetic Polypeptide
ATVKFTYRGEEKQVDISKIKHVRCWGQWIWFIYDKGGGAKGRGGVSEKGAPKELLQMLEKQGSEQKLISEED

SEQ ID NO: 118 - protein, 72 aa, synthetic construct
  REGION 1..72: note = Synthetic Polypeptide
ATVKFTYRGEEKQVGISRIRSVHRIGQWIKFWYDEGSGAYGRGYVSEKDAPKELLQMLEKRGSEQKLISEED

SEQ ID NO: 119 - protein, 72 aa, synthetic construct
  REGION 1..72: note = Synthetic Polypeptide
TTVKFTYRGEEKQVGISRIKSVHRIGQWIKFWYDKGSGAYGRGYVSEKDAPKELLQMLEKRGSEQKLISEED

SEQ ID NO: 120 - protein, 72 aa, synthetic construct
  REGION 1..72: note = Synthetic Polypeptide
AIVKFTYRGEEKQVGISRIKSVHRIGQWIKFWYGEGSGAYGRGYVSEKDAPKELLQMLEKRGSEQKLISEED

SEQ ID NO: 121 - protein, 72 aa, synthetic construct
  REGION 1..72: note = Synthetic Polypeptide
ATVKFTYRGGEKQVGISRIKSVHRIGQWIKFRYDEGSGAYGRGYVSEKDAPKELLQMLEKRGSEQKLISEED

SEQ ID NO: 122 - protein, 72 aa, synthetic construct
  REGION 1..72: note = Synthetic Polypeptide
ATVKFTYRGKEKQVGISRIKSVHRIGQWIKFRYDEGSGAYGRGYVSEKDAPKELLQMLEKRGSEQKLISEED

SEQ ID NO: 123 - protein, 72 aa, synthetic construct
  REGION 1..72: note = Synthetic Polypeptide
ATVKFTYRGEEKQVDISRIKHVRRWGRWIWFIYEEGGGAKGRGGVSEKDAPKELLQMLEKQGSEQKLISEED

SEQ ID NO: 124 - protein, 72 aa, synthetic construct
  REGION 1..72: note = Synthetic Polypeptide
ATVKFTYRGEEKQVDISKIKHVRRWGRWIWFIYEEGGGAKGRGGVSEKDAPKELLQMLEKQGSEQKLISEED

SEQ ID NO: 125 - protein, 72 aa, synthetic construct
  REGION 1..72: note = Synthetic Polypeptide
ATVKFTYRGEEKQVDISKIKHVRRWGRWVWFIYEEGGGAKGRGGVSEKDAPKELLQMLEKQGSEQKLISEED

SEQ ID NO: 126 - protein, 72 aa, synthetic construct
  REGION 1..72: note = Synthetic Polypeptide
ATVKFTYRGEEKQVDISKVKHVRRWGRWIWFIYEEGGGAKGRGGVSEKDAPKELLQMLEKQGSEQKLISEED

SEQ ID NO: 127 - protein, 72 aa, synthetic construct
  REGION 1..72: note = Synthetic Polypeptide
ATVKFTYRGEEKQVDISKIKHVRRWGRWIWFIYEEGGGAKGRGGVSEKDAPKELLRMLEKQGSEQKLISEED

SEQ ID NO: 128 - protein, 72 aa, synthetic construct
  REGION 1..72: note = Synthetic Polypeptide
ATVKFTYRGEEKQVDISKIKHVRRWGRWIWFIYEEGGGAKGRGGVSGKDAPKELLQMLEKQGSEQKLISEED

SEQ ID NO: 129 - protein, 68 aa, synthetic construct; identical to SEQ ID NO: 35
  REGION 1..68: note = Synthetic Polypeptide

SEQ ID NO: 130 - protein, 60 aa, synthetic construct; identical to SEQ ID NO: 36
  REGION 1..60: note = Synthetic Polypeptide

SEQ ID NO: 131 - protein, 72 aa, synthetic construct; identical to SEQ ID NO: 37
  REGION 1..72: note = Synthetic Polypeptide

SEQ ID NO: 132 - protein, 72 aa, synthetic construct; identical to SEQ ID NO: 38
  REGION 1..72: note = Synthetic Polypeptide

SEQ ID NO: 133 - protein, 72 aa, synthetic construct; identical to SEQ ID NO: 39
  REGION 1..72: note = Synthetic Polypeptide

SEQ ID NO: 134 - protein, 72 aa, synthetic construct; identical to SEQ ID NO: 40
  REGION 1..72: note = Synthetic Polypeptide

SEQ ID NO: 135 - protein, 72 aa, synthetic construct; identical to SEQ ID NO: 41
  REGION 1..72: note = Synthetic Polypeptide

SEQ ID NO: 136 - protein, 72 aa, synthetic construct; identical to SEQ ID NO: 42
  REGION 1..72: note = Synthetic Polypeptide

SEQ ID NO: 137 - protein, 72 aa, synthetic construct; identical to SEQ ID NO: 43
  REGION 1..72: note = Synthetic Polypeptide

SEQ ID NO: 138 - protein, 72 aa, synthetic construct; identical to SEQ ID NO: 44
  REGION 1..72: note = Synthetic Polypeptide

SEQ ID NO: 139 - protein, 72 aa, synthetic construct; identical to SEQ ID NO: 45
  REGION 1..72: note = Synthetic Polypeptide

SEQ ID NO: 140 - protein, 72 aa, synthetic construct; identical to SEQ ID NO: 27
  REGION 1..72: note = Synthetic Polypeptide

SEQ ID NO: 141 - protein, 72 aa, synthetic construct; identical to SEQ ID NO: 46
  REGION 1..72: note = Synthetic Polypeptide

SEQ ID NO: 142 - protein, 72 aa, synthetic construct; identical to SEQ ID NO: 47
  REGION 1..72: note = Synthetic Polypeptide

SEQ ID NO: 143 - protein, 72 aa, synthetic construct; identical to SEQ ID NO: 48
  REGION 1..72: note = Synthetic Polypeptide

SEQ ID NO: 144 - protein, 72 aa, synthetic construct; identical to SEQ ID NO: 49
  REGION 1..72: note = Synthetic Polypeptide

SEQ ID NO: 145 - protein, 72 aa, synthetic construct; identical to SEQ ID NO: 50
  REGION 1..72: note = Synthetic Polypeptide

SEQ ID NO: 146 - protein, 72 aa, synthetic construct; identical to SEQ ID NO: 51
  REGION 1..72: note = Synthetic Polypeptide

SEQ ID NO: 147 - protein, 72 aa, synthetic construct; identical to SEQ ID NO: 52
  REGION 1..72: note = Synthetic Polypeptide

SEQ ID NO: 148 - protein, 72 aa, synthetic construct; identical to SEQ ID NO: 53
  REGION 1..72: note = Synthetic Polypeptide

SEQ ID NO: 149 - protein, 72 aa, synthetic construct; identical to SEQ ID NO: 54
  REGION 1..72: note = Synthetic Polypeptide

SEQ ID NO: 150 - protein, 72 aa, synthetic construct; identical to SEQ ID NO: 55
  REGION 1..72: note = Synthetic Polypeptide

SEQ ID NO: 151 - protein, 72 aa, synthetic construct; identical to SEQ ID NO: 56
  REGION 1..72: note = Synthetic Polypeptide

SEQ ID NO: 152 - protein, 72 aa, synthetic construct; identical to SEQ ID NO: 57
  REGION 1..72: note = Synthetic Polypeptide

SEQ ID NO: 153 - protein, 72 aa, synthetic construct; identical to SEQ ID NO: 58
  REGION 1..72: note = Synthetic Polypeptide

SEQ ID NO: 154 - protein, 72 aa, synthetic construct; identical to SEQ ID NO: 59
  REGION 1..72: note = Synthetic Polypeptide

SEQ ID NO: 155 - protein, 72 aa, synthetic construct; identical to SEQ ID NO: 60
  REGION 1..72: note = Synthetic Polypeptide

SEQ ID NO: 156 - protein, 72 aa, synthetic construct; identical to SEQ ID NO: 61
  REGION 1..72: note = Synthetic Polypeptide

SEQ ID NO: 157 - protein, 72 aa, synthetic construct; identical to SEQ ID NO: 62
  REGION 1..72: note = Synthetic Polypeptide

SEQ ID NO: 158 - protein, 72 aa, synthetic construct; identical to SEQ ID NO: 63
  REGION 1..72: note = Synthetic Polypeptide

SEQ ID NO: 159 - protein, 72 aa, synthetic construct; identical to SEQ ID NO: 64
  REGION 1..72: note = Synthetic Polypeptide

SEQ ID NO: 160 - protein, 72 aa, synthetic construct; identical to SEQ ID NO: 65
  REGION 1..72: note = Synthetic Polypeptide

SEQ ID NO: 161 - protein, 72 aa, synthetic construct; identical to SEQ ID NO: 66
  REGION 1..72: note = Synthetic Polypeptide

SEQ ID NO: 162 - protein, 72 aa, synthetic construct; identical to SEQ ID NO: 67
  REGION 1..72: note = Synthetic Polypeptide

SEQ ID NO: 163 - protein, 72 aa, synthetic construct; identical to SEQ ID NO: 68
  REGION 1..72: note = Synthetic Polypeptide

SEQ ID NO: 164 - protein, 72 aa, synthetic construct; identical to SEQ ID NO: 69
  REGION 1..72: note = Synthetic Polypeptide

SEQ ID NO: 165 - protein, 72 aa, synthetic construct; identical to SEQ ID NO: 32
  REGION 1..72: note = Synthetic Polypeptide

SEQ ID NO: 166 - protein, 72 aa, synthetic construct; identical to SEQ ID NO: 70
  REGION 1..72: note = Synthetic Polypeptide

SEQ ID NO: 167 - protein, 72 aa, synthetic construct; identical to SEQ ID NO: 71
  REGION 1..72: note = Synthetic Polypeptide

SEQ ID NO: 168 - protein, 72 aa, synthetic construct; identical to SEQ ID NO: 72
  REGION 1..72: note = Synthetic Polypeptide

SEQ ID NO: 169 - protein, 72 aa, synthetic construct; identical to SEQ ID NO: 73
  REGION 1..72: note = Synthetic Polypeptide

SEQ ID NO: 170 - protein, 72 aa, synthetic construct; identical to SEQ ID NO: 74
  REGION 1..72: note = Synthetic Polypeptide

SEQ ID NO: 171 - protein, 72 aa, synthetic construct; identical to SEQ ID NO: 75
  REGION 1..72: note = Synthetic Polypeptide

SEQ ID NO: 172 - protein, 72 aa, synthetic construct; identical to SEQ ID NO: 76
  REGION 1..72: note = Synthetic Polypeptide

SEQ ID NO: 173 - protein, 72 aa, synthetic construct; identical to SEQ ID NO: 77
  REGION 1..72: note = Synthetic Polypeptide

SEQ ID NO: 174 - protein, 72 aa, synthetic construct; identical to SEQ ID NO: 78
  REGION 1..72: note = Synthetic Polypeptide

SEQ ID NO: 175 - protein, 72 aa, synthetic construct; identical to SEQ ID NO: 79
  REGION 1..72: note = Synthetic Polypeptide

SEQ ID NO: 176 - protein, 72 aa, synthetic construct; identical to SEQ ID NO: 80
  REGION 1..72: note = Synthetic Polypeptide

SEQ ID NO: 177 - protein, 72 aa, synthetic construct; identical to SEQ ID NO: 25
  REGION 1..72: note = Synthetic Polypeptide

SEQ ID NO: 178 - protein, 72 aa, synthetic construct; identical to SEQ ID NO: 81
  REGION 1..72: note = Synthetic Polypeptide

SEQ ID NO: 179 - protein, 72 aa, synthetic construct; identical to SEQ ID NO: 82
  REGION 1..72: note = Synthetic Polypeptide

SEQ ID NO: 180 - protein, 72 aa, synthetic construct; identical to SEQ ID NO: 83
  REGION 1..72: note = Synthetic Polypeptide

SEQ ID NO: 181 - protein, 72 aa, synthetic construct; identical to SEQ ID NO: 84
  REGION 1..72: note = Synthetic Polypeptide

SEQ ID NO: 182 - protein, 72 aa, synthetic construct; identical to SEQ ID NO: 85
  REGION 1..72: note = Synthetic Polypeptide

SEQ ID NO: 183 - protein, 72 aa, synthetic construct; identical to SEQ ID NO: 86
  REGION 1..72: note = Synthetic Polypeptide

SEQ ID NO: 184 - protein, 72 aa, synthetic construct; identical to SEQ ID NO: 87
  REGION 1..72: note = Synthetic Polypeptide

SEQ ID NO: 185 - protein, 72 aa, synthetic construct; identical to SEQ ID NO: 88
  REGION 1..72: note = Synthetic Polypeptide

SEQ ID NO: 186 - protein, 72 aa, synthetic construct; identical to SEQ ID NO: 89
  REGION 1..72: note = Synthetic Polypeptide

SEQ ID NO: 187 - protein, 69 aa, synthetic construct; identical to SEQ ID NO: 90
  REGION 1..69: note = Synthetic Polypeptide

SEQ ID NO: 188 - protein, 69 aa, synthetic construct; identical to SEQ ID NO: 91
  REGION 1..69: note = Synthetic Polypeptide

SEQ ID NO: 189 - protein, 69 aa, synthetic construct; identical to SEQ ID NO: 92
  REGION 1..69: note = Synthetic Polypeptide

SEQ ID NO: 190 - protein, 69 aa, synthetic construct; identical to SEQ ID NO: 93
  REGION 1..69: note = Synthetic Polypeptide

SEQ ID NO: 191 - protein, 69 aa, synthetic construct; identical to SEQ ID NO: 94
  REGION 1..69: note = Synthetic Polypeptide

SEQ ID NO: 192 - protein, 72 aa, synthetic construct; identical to SEQ ID NO: 95
  REGION 1..72: note = Synthetic Polypeptide

SEQ ID NO: 193 - protein, 72 aa, synthetic construct; identical to SEQ ID NO: 96
  REGION 1..72: note = Synthetic Polypeptide

SEQ ID NO: 194 - protein, 72 aa, synthetic construct; identical to SEQ ID NO: 97
  REGION 1..72: note = Synthetic Polypeptide

SEQ ID NO: 195 - protein, 72 aa, synthetic construct; identical to SEQ ID NO: 98
  REGION 1..72: note = Synthetic Polypeptide

SEQ ID NO: 196 - protein, 72 aa, synthetic construct; identical to SEQ ID NO: 99
  REGION 1..72: note = Synthetic Polypeptide

SEQ ID NO: 197 - protein, 72 aa, synthetic construct; identical to SEQ ID NO: 100
  REGION 1..72: note = Synthetic Polypeptide

SEQ ID NO: 198 - protein, 72 aa, synthetic construct; identical to SEQ ID NO: 101
  REGION 1..72: note = Synthetic Polypeptide

SEQ ID NO: 199 - protein, 72 aa, synthetic construct; identical to SEQ ID NO: 102
  REGION 1..72: note = Synthetic Polypeptide

SEQ ID NO: 200 - protein, 72 aa, synthetic construct; identical to SEQ ID NO: 103
  REGION 1..72: note = Synthetic Polypeptide

SEQ ID NO: 201 - protein, 72 aa, synthetic construct; identical to SEQ ID NO: 104
  REGION 1..72: note = Synthetic Polypeptide

SEQ ID NO: 202 - protein, 72 aa, synthetic construct; identical to SEQ ID NO: 105
  REGION 1..72: note = Synthetic Polypeptide

SEQ ID NO: 203 - protein, 72 aa, synthetic construct; identical to SEQ ID NO: 106
  REGION 1..72: note = Synthetic Polypeptide

SEQ ID NO: 204 - protein, 72 aa, synthetic construct; identical to SEQ ID NO: 107
  REGION 1..72: note = Synthetic Polypeptide

SEQ ID NO: 205 - protein, 72 aa, synthetic construct; identical to SEQ ID NO: 108
  REGION 1..72: note = Synthetic Polypeptide

SEQ ID NO: 206 - protein, 72 aa, synthetic construct; identical to SEQ ID NO: 109
  REGION 1..72: note = Synthetic Polypeptide

SEQ ID NO: 207 - protein, 72 aa, synthetic construct; identical to SEQ ID NO: 110
  REGION 1..72: note = Synthetic Polypeptide

SEQ ID NO: 208 - protein, 72 aa, synthetic construct; identical to SEQ ID NO: 111
  REGION 1..72: note = Synthetic Polypeptide

SEQ ID NO: 209 - protein, 72 aa, synthetic construct; identical to SEQ ID NO: 112
  REGION 1..72: note = Synthetic Polypeptide

SEQ ID NO: 210 - protein, 72 aa, synthetic construct; identical to SEQ ID NO: 113
  REGION 1..72: note = Synthetic Polypeptide

SEQ ID NO: 211 - protein, 72 aa, synthetic construct; identical to SEQ ID NO: 114
  REGION 1..72: note = Synthetic Polypeptide

SEQ ID NO: 212 - protein, 72 aa, synthetic construct; identical to SEQ ID NO: 115
  REGION 1..72: note = Synthetic Polypeptide

SEQ ID NO: 213 - protein, 72 aa, synthetic construct; identical to SEQ ID NO: 116
  REGION 1..72: note = Synthetic Polypeptide

SEQ ID NO: 214 - protein, 72 aa, synthetic construct; identical to SEQ ID NO: 117
  REGION 1..72: note = Synthetic Polypeptide

SEQ ID NO: 215 - protein, 72 aa, synthetic construct; identical to SEQ ID NO: 118
  REGION 1..72: note = Synthetic Polypeptide

SEQ ID NO: 216 - protein, 72 aa, synthetic construct; identical to SEQ ID NO: 119
  REGION 1..72: note = Synthetic Polypeptide

SEQ ID NO: 217 - protein, 72 aa, synthetic construct; identical to SEQ ID NO: 120
  REGION 1..72: note = Synthetic Polypeptide

SEQ ID NO: 218 - protein, 72 aa, synthetic construct; identical to SEQ ID NO: 121
  REGION 1..72: note = Synthetic Polypeptide

SEQ ID NO: 219 - protein, 72 aa, synthetic construct; identical to SEQ ID NO: 122
  REGION 1..72: note = Synthetic Polypeptide

SEQ ID NO: 220 - protein, 72 aa, synthetic construct; identical to SEQ ID NO: 123
  REGION 1..72: note = Synthetic Polypeptide

SEQ ID NO: 221 - protein, 72 aa, synthetic construct; identical to SEQ ID NO: 124
  REGION 1..72: note = Synthetic Polypeptide

SEQ ID NO: 222 - protein, 72 aa, synthetic construct; identical to SEQ ID NO: 125
  REGION 1..72: note = Synthetic Polypeptide

SEQ ID NO: 223 - protein, 72 aa, synthetic construct; identical to SEQ ID NO: 126
  REGION 1..72: note = Synthetic Polypeptide

SEQ ID NO: 224 - protein, 72 aa, synthetic construct; identical to SEQ ID NO: 127
  REGION 1..72: note = Synthetic Polypeptide

SEQ ID NO: 225 - protein, 72 aa, synthetic construct; identical to SEQ ID NO: 128
  REGION 1..72: note = Synthetic Polypeptide

SEQ ID NO: 226 - protein, 72 aa, synthetic construct
  REGION 1..72: note = Synthetic Polypeptide
ATVRFTYRGEEKQVDISKIKYVRRWGQYIWFGYDGGGGARGYGYVSERDAPKELLQMLEEQGSEQKLISEED

SEQ ID NO: 227 - protein, 72 aa, synthetic construct
  REGION 1..72: note = Synthetic Polypeptide
ATVRFTYQGEEKQVDISKIKHVRRWGRYIWFGYDEGGGARGHGYVSEKDAPKELLQMLEKQGSEQKLISEED

SEQ ID NO: 228 - protein, 72 aa, synthetic construct
  REGION 1..72: note = Synthetic Polypeptide
ATVQFTYQGEERQVDISKIRHVRRWGRWIWFIYGEGGGAKGWGGVSAKDAPKELLQMLEKQGSEQKLISEED

SEQ ID NO: 229 - protein, 72 aa, synthetic construct
  REGION 1..72: note = Synthetic Polypeptide
ATVKFTYQGGEKQVDISKIKHVRRWGQWIWFIYDEGGGAKGWGGVSGKDAPKELLQMLEKRGSEQKLISEED

SEQ ID NO: 230 - protein, 72 aa, synthetic construct
  REGION 1..72: note = Synthetic Polypeptide
ATVKFTYRGEEKQVDISKIKYVRRWGQYIWFGYDEGGGARGYGYVSERDAPKELLQMLEKQGSEQKLISEED

SEQ ID NO: 231 - protein, 72 aa, synthetic construct
  REGION 1..72: note = Synthetic Polypeptide
ATVKFTYRGEEKQVDISKIKHVRRWGQYIWFGYDEGGGARGYGYVSEKDAPKGLLQMLEKQGSEQKLISEED

SEQ ID NO: 232 - protein, 72 aa, synthetic construct
  REGION 1..72: note = Synthetic Polypeptide
ATVKFTYRGEEKQVDISKIKHVRRWGQWIWFIYDEGGGAKGWGGVSERDAPKGLLQMLEKQGSEQKLISEED

SEQ ID NO: 233 - protein, 72 aa, synthetic construct
  REGION 1..72: note = Synthetic Polypeptide
AAVKFTYQGEEKQVDISKIKYVWRWGRWIWFRYDEGGGAHGIGHVSEKDVPKELLQMLEKQGSEQKLISEED

SEQ ID NO: 234 - protein, 72 aa, synthetic construct
  REGION 1..72: note = Synthetic Polypeptide
ATVRFTYRGEEKQVDISRIKYVRRWGQYIWLGYDGGGGARGYGYVSEKGAPKELLQMLEKQGSEQKLISEED

SEQ ID NO: 235 - protein, 72 aa, synthetic construct
  REGION 1..72: note = Synthetic Polypeptide
ATVKFTYRGEEKQVDISKIKYVRRWGQYIWFGYGEGGGARGYGYVSEKDAPKELLQMLKKQGSEQKLISEED

SEQ ID NO: 236 - protein, 72 aa, synthetic construct
  REGION 1..72: note = Synthetic Polypeptide
AAVKFTYQGEEKQVDTSKIKHVRRWGRYIWFGYDEGGGARGHGYVSEKDAPKELLQMLEKQGSEQKLISEED

SEQ ID NO: 237 - protein, 72 aa, synthetic construct
  REGION 1..72: note = Synthetic Polypeptide
ATVKFTYRGEEGQVDISKVKYVWRWGQWIWFRYDGGGGAHGIGYVSEKDTPKELLQMLEKQGSEQKLISEED

SEQ ID NO: 238 - protein, 72 aa, synthetic construct
  REGION 1..72: note = Synthetic Polypeptide
ATVKFTYQGEEKQVGISKIRYVRRWGQYIWFGYDEGGGTRGYGYVSERDAPKELLQMLERRGSEQKLISEED

SEQ ID NO: 239 - protein, 72 aa, synthetic construct
  REGION 1..72: note = Synthetic Polypeptide
ATVKFTYQGEEKQVDISKIKHVRRWGQWIWFIYDEGGGARGRGYVSEKGAPEELLQMLGKQGSEQKLISEED

SEQ ID NO: 240 - protein, 72 aa, synthetic construct
  REGION 1..72: note = Synthetic Polypeptide
ATVKFTYHGEGKQVDISKIKYVRRWGRYIWFGYDEGGGARGYGYVSEKGAPEELLQMLEKQGSEQKLISEED

SEQ ID NO: 241 - protein, 72 aa, synthetic construct
  REGION 1..72: note = Synthetic Polypeptide
ATVKFTYRGEEKQVDISKIKYVRRWGRYIWFGYDEGGGARGYGYVSEKDAPKELLQMLEKQGSEQKLISEED

SEQ ID NO: 242 - protein, 72 aa, synthetic construct
  REGION 1..72: note = Synthetic Polypeptide
ATVKFTYQGEEKQVDISKIKHVRRWGQWIWFIYDEGGGAKGWGGVSGKDAPKELLQMLEKQGSEQKLISEED

SEQ ID NO: 243 - protein, 72 aa, synthetic construct
  REGION 1..72: note = Synthetic Polypeptide
ATVKFTYQGEEKQVDISKIKYVRRWGQYIWFGYGEGGGARGYGHVSERDAPKELLQMLEKQGSEQKLISEED

SEQ ID NO: 244 - protein, 72 aa, synthetic construct
  REGION 1..72: note = Synthetic Polypeptide
ATVKFTYRGEEKQVAISKIKYVRRWGQHIWFGYDKGGGAHGIGYVSERDAPKELLQMLDEQGSEQKLISEED

SEQ ID NO: 245 - protein, 72 aa, synthetic construct
  REGION 1..72: note = Synthetic Polypeptide
ATVKFTYRGEEKRVDISKIKHVRRWGQWIWFIYDGGGGAKGWGGVSEKDAPKELLQMLEEQGSEQKLISEED

SEQ ID NO: 246 - protein, 72 aa, synthetic construct
  REGION 1..72: note = Synthetic Polypeptide
ATVKFTYRGEEKQVDISKIKYVRRWGQYIWFGYDEGGGARGYGYVSEKDAPEELLQMLEKHGSEQKLISEED

SEQ ID NO: 247 - protein, 72 aa, synthetic construct
  REGION 1..72: note = Synthetic Polypeptide
ATVRFTYHGEEKQVDISKIKYVRRWGQWIWFIYDEGGGANGKGSVSEKDAPKELLQMLEKQGSEQKLISEED

SEQ ID NO: 248 - protein, 72 aa, synthetic construct
  REGION 1..72: note = Synthetic Polypeptide
ATVKFTYRGEEKQVDISKIKYVRRWGQYIWFGYDEGGGARGHGYVSENDAPKELLQMLEKQGSEQKLISEED

SEQ ID NO: 249 - protein, 72 aa, synthetic construct
  REGION 1..72: note = Synthetic Polypeptide
ATVEFTYRGEEKQVDISKIKYVRRWGQYIWFGYDGGGGARGYGYVSEKDAPKELLQMLEEQGSEQKLISEED

SEQ ID NO: 250 - protein, 72 aa, synthetic construct
  REGION 1..72: note = Synthetic Polypeptide
ATVKFTYRGEEKQVGISKIKYVRRWGQYIWFGYDEGGGARGYGYVSEKDAPKELLQMLDKQGSEQKLISEED

SEQ ID NO: 251 - protein, 72 aa, synthetic construct
  REGION 1..72: note = Synthetic Polypeptide
ATVKFTYRGEEKQVDISKIKHVRRWGQWIWFIYDGGGGAKGWGGVSEKDAPKELLQMLEKQGSEQKLISEED

SEQ ID NO: 252 - protein, 72 aa, synthetic construct
  REGION 1..72: note = Synthetic Polypeptide
ATVKFTYRGEEKQVDISKIKHVRRWGQWIWFIYDEGGGAKGWGGVSEKDAPKELLQMLEKQGSEQKLISEED

SEQ ID NO: 253 - protein, 60 aa, synthetic construct
  REGION 1..60: note = Synthetic Polypeptide
AVVRFTYRGEEKQVDISKIKYVRRWGQYIWFGYDEGGGARGYGYVSEKDAPKELLRMLEK

SEQ ID NO: 254 - protein, 72 aa, synthetic construct
  REGION 1..72: note = Synthetic Polypeptide
ATVKFTYQGEEKRVDISKIKYVRRWGQYIWFGYDGGGGARGYGHVSEKDAPRELLQMLEKRGSEQKLISEED

SEQ ID NO: 255 - protein, 72 aa, synthetic construct
  REGION 1..72: note = Synthetic Polypeptide
ATVKFTYQGEEKQVDISKIKYVWRWGQWIWFHYDEGGGARGYGYVSEKDAPKELLQMLGKRGSEQKLISEED

SEQ ID NO: 256 - protein, 72 aa, synthetic construct
  REGION 1..72: note = Synthetic Polypeptide
ATVKFTYQGEEKQVDISKIKYVRRWGQYIWFGYDGGGGARGYGHVSEKDAPKELLQMLGGRGSEQKLISEED

SEQ ID NO: 257 - protein, 72 aa, synthetic construct
  REGION 1..72: note = Synthetic Polypeptide
ATVKFTYRGEEKQVDISKIKYVRRWGQYIWFGYDGGGGARGYGHVSEKDAPKELLQMLEEQGSEQKLISEED

SEQ ID NO: 258 - protein, 72 aa, synthetic construct
  REGION 1..72: note = Synthetic Polypeptide
ATVRFTYQGEEKQVDISKTKHVRRWGQWIWFIYDEAGGAHGRGRVSEKDAPKELLQMLEKQGSEQKLISEED

SEQ ID NO: 259 - protein, 72 aa, synthetic construct
  REGION 1..72: note = Synthetic Polypeptide
ATVKFTYRGEEKQVDISKIKYVRRWGQYIWFGYDEGGGARGYGYVSETDAPEKLLQMLEKQGSEQKLISEED

SEQ ID NO: 260 - protein, 72 aa, synthetic construct
  REGION 1..72: note = Synthetic Polypeptide
ATVKFTYRGEEKQVDISKIKHVRRWGQWIWFIYDGGGGTKGWGGVSEKDAPKELLQMLEKQGSEQKLISEED

SEQ ID NO: 261 - protein, 72 aa, synthetic construct
  REGION 1..72: note = Synthetic Polypeptide
ATVKFTYQGEEKQVDISKIKHVRRWGRWIWFIYDEGGGAKGWGGVSGRDAPKELLQMLEKQGSEQKLISEED

SEQ ID NO: 262 - protein, 72 aa, synthetic construct
  REGION 1..72: note = Synthetic Polypeptide
ATVKFTYQGEEKQVGISRIKYVRRRGQYIWFGYDKGGGARGYGYVSEKDAPKELLQMLEKQGSEQKLISEED

SEQ ID NO: 263 - protein, 72 aa, synthetic construct
  REGION 1..72: note = Synthetic Polypeptide
ATVRFTYQGEEKQVDISKIKHVRRWGQWIWFIYDEGGGAKGRGGVSEKDAPKELLQMLEKQGSEQKLISEED

SEQ ID NO: 264 - protein, 72 aa, synthetic construct
  REGION 1..72: note = Synthetic Polypeptide
ATVKFTYRGEEKQVDISKIKYVRRWGQYIWFGYDEGGGARGYGYVSEKDAPKGLLQMLEKRGSEQKLISEED

SEQ ID NO: 265 - protein, 72 aa, synthetic construct
  REGION 1..72: note = Synthetic Polypeptide
ATVKFTYQGEEKQVDISKIKYVRRWGQRISFIYDEGGGARGYGRVSEKDAPKELLQLLEKQGSEQKLISEED

SEQ ID NO: 266 - protein, 72 aa, synthetic construct
  REGION 1..72: note = Synthetic Polypeptide
ATVKFTYRGEEEQVDISKIKYVWRWGQWIWLRYDEGGGAHGIGYVSRKDAPKELLQMLEKQGSEQKLISEED

SEQ ID NO: 267 - protein, 72 aa, synthetic construct
  REGION 1..72: note = Synthetic Polypeptide
ATVRFTYQGEERRVDISKIKYVRRWGQHIWFGYDEGGGARGYGYVNEKGAPRELLRMLEKRGSEQKLISEED

SEQ ID NO: 268 - protein, 72 aa, synthetic construct
  REGION 1..72: note = Synthetic Polypeptide
ATVKFTYQGEEKQVDISKIKHVRRWGRWIWFIYDEGGGAKGRGGVSEKDAPKELLQMLEKQGSEQKLISEED

SEQ ID NO: 269 - protein, 72 aa, synthetic construct
  REGION 1..72: note = Synthetic Polypeptide
ATVKFTYQGEEKQVDISEIKYVRRRGQYIWFGYDEGGGARGYGYVSGKDAPKELLQMLERRGSEQKLISEED

SEQ ID NO: 270 - protein, 72 aa, synthetic construct
  REGION 1..72: note = Synthetic Polypeptide
ATVKFTYQGKEGQVAISKIKYVRRWGQYIWFGYDEGGGARGYGYVSEKDAPKELLQMLGKQGSEQKLISEED

SEQ ID NO: 271 - protein, 72 aa, synthetic construct
  REGION 1..72: note = Synthetic Polypeptide
ATVRFTYQGEEKQVDISKIKYVRRWGQYIWFGYDGGGGARGYGYVSKKDAPKELLQMLERQGSEQKLISEED

SEQ ID NO: 272 - protein, 72 aa, synthetic construct
  REGION 1..72: note = Synthetic Polypeptide
ATVKFTYQGEEKQVDISKIKYVWRWGRWIWFRYDEGGGAHGIGHVSEKGAPKELLRMLEKQGSEQKLISEED

SEQ ID NO: 273 - protein, 72 aa, synthetic construct
  REGION 1..72: note = Synthetic Polypeptide
ATVKFTYQGEEKQVDISKIKHVRRWGRYIWFGYDEGGGARGYGYVSEKAAPKGLLQMLGKQGSEQKLISEED

SEQ ID NO: 274 - protein, 72 aa, synthetic construct
  REGION 1..72: note = Synthetic Polypeptide
ATVKFTYRGEEKQVDISKIKYVRRWGQYIWFGYDEGGGARGYGYVSGKDAPKELLQMLEKQGSEQKLISEED

SEQ ID NO: 275 - protein, 60 aa, synthetic construct
  REGION 1..60: note = Synthetic Polypeptide
ATVRFTYQGEEKQVDISRIKIVYRWGQRISFIYDKGGGARGYGRVSEKDAPKELLQMLEK

SEQ ID NO: 276 - protein, 72 aa, synthetic construct
  REGION 1..72: note = Synthetic Polypeptide
ATVRFTYRGGEKQVDISKIKYVRRWGQYVWFGYDKGGGVRGYGYVSEKDAPRELLQMLEKQGSEQKLISEED

SEQ ID NO: 277 - protein, 69 aa, synthetic construct
  REGION 1..69: note = Synthetic Polypeptide
ATVKFTYRGEEKQVDISRIRSVSRWGQAIVFRYDEGGGAKGKGSVSEKDAPKELLQMLGKAGIRTKAYF

SEQ ID NO: 278 - protein, 69 aa, synthetic construct
  REGION 1..69: note = Synthetic Polypeptide
ATVKFTYQGEEKQVGISKIKHVRRWGQWIWFIYDEGGGAKGRGSVSERDAPKELLQMLEKAGIRTKAYF

SEQ ID NO: 279 - protein, 69 aa, synthetic construct
  REGION 1..69: note = Synthetic Polypeptide
ATVKFTYQGEEKQVDISKIKYVRRWGQYIWFGYDEGGGARGYGYVSEKDAPKGLLQMLEKARIRTKAYF

SEQ ID NO: 280 - protein, 69 aa, synthetic construct
  REGION 1..69: note = Synthetic Polypeptide
ATVKFTYRGEEKRVDISKIKYVRRWGQYIWFGYDEGGGARGYGYVSGRDAPRELLQMLEKARIRTKAYF

SEQ ID NO: 281 - protein, 69 aa, synthetic construct
  REGION 1..69: note = Synthetic Polypeptide
ATVQFTYQGGEKQVDISKIKYVRRWGRYIWLGYDEGGGARGHGYVSEKDAPKELLQMLEKARIRTKAYF

SEQ ID NO: 282 - protein, 69 aa, synthetic construct
  REGION 1..69: note = Synthetic Polypeptide
ATVEFTYQGEEKQVDISKIKYVRRWGQYIWFGYDEGGGARGYGYVSEKDAPKELLQILEKARIRTKAYF

SEQ ID NO: 283 - protein, 69 aa, synthetic construct
  REGION 1..69: note = Synthetic Polypeptide
ATVKFTYQGEEKQVDISKIKYVRRWGQYLWFGYDGGGGARGYGYVSEKDAPKELLQMLERARIRTKAYF

SEQ ID NO: 284 - protein, 69 aa, synthetic construct
  REGION 1..69: note = Synthetic Polypeptide
ATVKFTYQGEERQVDISKVKHVRRWGQWVWFIYDEGGGAKGWGGVSEKDAPTELLQMLEKARIRTKAYF

SEQ ID NO: 285 - protein, 69 aa, synthetic construct
  REGION 1..69: note = Synthetic Polypeptide
ATVKFTYQGEEKQVDISRIKSAFRWGQAIIFRYDEGGGAKGKGSVSEKDAPKELLQMLEKARIRTKAYF

SEQ ID NO: 286 - protein, 69 aa, synthetic construct
  REGION 1..69: note = Synthetic Polypeptide
ATVEFTYRGEEKQVDISKIKYVRRWGQYIWFGYDEGGGARGYGYVSEKDAPKELLQMLEKAGIRTKAYF

SEQ ID NO: 287 - protein, 69 aa, synthetic construct
  REGION 1..69: note = Synthetic Polypeptide
ATVKFTYQGEEKQVDISKIKYARRWGQYIWFGYDEGGGARGYGYVSEEDAPKELLQMLEKARIRTKAYF

SEQ ID NO: 288 - protein, 69 aa, synthetic construct
  REGION 1..69: note = Synthetic Polypeptide
ATVKFTYRGEEKQVDISKIKSVSRWGQAIIFRYDEGGGAKGKGSVSEKDAPKELLQMLEKARIRTKAYF

SEQ ID NO: 289 - protein, 69 aa, synthetic construct
  REGION 1..69: note = Synthetic Polypeptide
ATVKFAYQGEERQVDISKIEYVRRWGQYIWFGYDEGGGARGYGYVSEKDAPKELLQMLEKARIRTKAYF

SEQ ID NO: 290 - protein, 69 aa, synthetic construct
  REGION 1..69: note = Synthetic Polypeptide
ATVKFTYQGEEKQVDVSKIKHVRRWGQWIWFIYDEGGGAKGWGGVSEKDAPKELLQMLEKARIRTKAYF

SEQ ID NO: 291 - protein, 69 aa, synthetic construct
  REGION 1..69: note = Synthetic Polypeptide
ATVKFTYQGEEKQVGIGKIKHVRRWGQYIWFGYDGGGGARGYGYVSEKDAPKGLLQMLEKARIRTKAYF

SEQ ID NO: 292 - protein, 69 aa, synthetic construct
  REGION 1..69: note = Synthetic Polypeptide
ATVKFTYQGEEKQVDISKIKYVWRWGQWIWFRYDEGGGAKGKGSVSEKDAPRELLQMLEKAGIRTKAYF

SEQ ID NO: 293 - protein, 69 aa, synthetic construct
  REGION 1..69: note = Synthetic Polypeptide
ATVKFTYRGEEKQVDISKIKYVRRWGQYIWFGYDGGGGARGYGHVSEKDAPKELLQMLEEARIRTKAYF

SEQ ID NO: 294 - protein, 69 aa, synthetic construct
  REGION 1..69: note = Synthetic Polypeptide
ATVKFTYQGEEKQVDIGKIKYVRRWGQYIWFGYDEGGGARGYGYVSEEDAPKELLQMLEKARIRTKAYF

SEQ ID NO: 295 - protein, 69 aa, synthetic construct
  REGION 1..69: note = Synthetic Polypeptide
ATVKFTYQGEEKQVDISKIKSVHRVGQWIKFWYDEGGGAYGRGYVSERDAPRELLQMLEEARIRTKAYF

SEQ ID NO: 296 - protein, 69 aa, synthetic construct
  REGION 1..69: note = Synthetic Polypeptide
ATVKFTYQGEEEQVAISKIKHVRRWGQWIWFRYDEGGGAHGIGYVSEKDAPKELLQMLEKARIRTKAYF

SEQ ID NO: 297 - protein, 69 aa, synthetic construct
  REGION 1..69: note = Synthetic Polypeptide
ATVKFTYQGEEKQVDISKIKYVWRWGQWIWFRYDEGGGAHGIGYMSEKDAPRELLQMLGKARIRTKAYF

SEQ ID NO: 298 - protein, 69 aa, synthetic construct
  REGION 1..69: note = Synthetic Polypeptide
ATVKFTYQGEEKQVEVSKIKYVRRWGQYIWFSYDEGGGARGYGYVSERDAPRELLQMLEKARIRTKAYF

SEQ ID NO: 299 - protein, 69 aa, synthetic construct
  REGION 1..69: note = Synthetic Polypeptide
ATVKFTYRGEEKQVDISKIKSVSRWGQAIIFRYDGGGGARGKGSVSEKDAPKELLQMLEKAGIRTKAYF

SEQ ID NO: 300 - protein, 69 aa, synthetic construct
  REGION 1..69: note = Synthetic Polypeptide
ATVKFTYQGEEKQVDISKIKHVRRWGQWIWFIYDEGGGAKGWGGVSEKNAPKELLQMLEKARIRTKAYF

SEQ ID NO: 301 - protein, 69 aa, synthetic construct
  REGION 1..69: note = Synthetic Polypeptide
ATVRFTYQGEEKQVDISKIKYVRRWGQYIWFGYDEGGGARGYGYVSEKDTPKELLQLLEKARIRTKAYF

SEQ ID NO: 302 - protein, 69 aa, synthetic construct; identical to SEQ ID NO: 29
  REGION 1..69: note = Synthetic Polypeptide

SEQ ID NO: 303 - protein, 69 aa, synthetic construct
  REGION 1..69: note = Synthetic Polypeptide
ATVKFTYQGEEKQVDISKIKYVWRWGQAIIFRYDEGGGAKGKGSVSEEDAPKELLQMLEKARIRTKAYF

SEQ ID NO: 304 - protein, 72 aa, synthetic construct
  REGION 1..72: note = Synthetic Polypeptide
ATVKFTYRGEEKQVGISKIKYVRRWGQYIWFGYDEGGGARGHGYVSEKDAPKELLQMLEKQGSEQKLISEED

SEQ ID NO: 305 - protein, 72 aa, synthetic construct
  REGION 1..72: note = Synthetic Polypeptide
AAVKFTYQGEEKQVDIGRTKYVWRWGQWIWFRYDEGGGARGYGCVGEKDAPRELLRVLEKQGSEQKLISEED

SEQ ID NO: 306 - protein, 72 aa, synthetic construct
  REGION 1..72: note = Synthetic Polypeptide
ATVKFTYRGEEKQVDTSRIKYVWRWGQWIWFRYDEGGGARGYGYVSEKDAPKELLQMLEKQGSEQKLISEED

SEQ ID NO: 307 - protein, 72 aa, synthetic construct
  REGION 1..72: note = Synthetic Polypeptide
ATVRFTYQGEERQVGISKIKYVRRRGQYIWFGYDEGGGVRGYGYVSEKGAPKELLRMLEKRGSEQKLISEED

SEQ ID NO: 308 - protein, 72 aa, synthetic construct
  REGION 1..72: note = Synthetic Polypeptide
ATVKFTYQGEEKQVDISKIKHVRRWGQWIWFIYDEAGGAHGRGRVSERGAPKELLQMLEKQGSEQKLISEED

SEQ ID NO: 309 - protein, 72 aa, synthetic construct
  REGION 1..72: note = Synthetic Polypeptide
ATVKFTYRGEEEQVGISRIKYVWRWGQWIWFRYDGGGGARGYGHVSDKDAPKELLQMLEKRGSEQKLISEED

SEQ ID NO: 310 - protein, 72 aa, synthetic construct
  REGION 1..72: note = Synthetic Polypeptide
ATVKFTYQGEEKQVDISRIKHVRRWGQWIWFIYDGAGGAHGRGRVSERGAPKELLQMLEKQGSEQKLISEED

SEQ ID NO: 311 - protein, 72 aa, synthetic construct
  REGION 1..72: note = Synthetic Polypeptide
ATVKFTYQGEEKQVDISKVKYVRRRGQYIWFGYDEGDGAYGRGHVSEKGAPKELLQMLKKQGSEQKLISEED

SEQ ID NO: 312 - protein, 72 aa, synthetic construct
  REGION 1..72: note = Synthetic Polypeptide
VTVKFTYQGEEKQVDISRIKHVRRWGQWIWFIYGKGGGAKGRGGVSEKDAPKELLQMLEKQGSEQKLISEED

SEQ ID NO: 313 - protein, 72 aa, synthetic construct
  REGION 1..72: note = Synthetic Polypeptide
AAVKFTYRGEEKQVDISKIKYVRRWGQYIWFGYDKGGGARGYGYVGEKGAPKELLQMLEKQGSEQKLISEED

SEQ ID NO: 314 - protein, 72 aa, synthetic construct
  REGION 1..72: note = Synthetic Polypeptide
ATVKFTYRGEEKQVDISRIKYVRRWGQYIWFGYDEGGGARGHGHVSEKEAPRELLQMLEKQGSEQKLISEED

SEQ ID NO: 315 - protein, 72 aa, synthetic construct
  REGION 1..72: note = Synthetic Polypeptide
ATVKFTYQGEEKQVDISKIKHVRRWGQWIWFIYDEAGGAHGRGRVSERGAPRELLQMLEKQGSEQKLISEED

SEQ ID NO: 316 - protein, 72 aa, synthetic construct
  REGION 1..72: note = Synthetic Polypeptide
ATVRFTYRGEERQVGISKIKYVRRWGQYIWFGYDEGGGARGYGYVSEKDAPKELLQMLDKQGSEQKLISEED

SEQ ID NO: 317 - protein, 72 aa, synthetic construct
  REGION 1..72: note = Synthetic Polypeptide
ATVKFTYQGEEKQVGISRIKYVRRRGQYIWFGYDKGGGARGHGYVGEKDAPKELLQMLGKQGSEQKLISEED

SEQ ID NO: 318 - protein, 72 aa, synthetic construct
  REGION 1..72: note = Synthetic Polypeptide
ATVKFTYQGGEKQVDISKIKYVRRWGQHIWFGYDEGGGARGYGYVSKKDAPKELLQMLEKRGSEQKLISEED

SEQ ID NO: 319 - protein, 72 aa, synthetic construct
  REGION 1..72: note = Synthetic Polypeptide
ATVKFTYRGEEGQVDISKVKYVWRWGQWIWFRYDGGGGAHGIGHVSEKDTPKELLQMLEKQGSEQKLISEED

SEQ ID NO: 320 - protein, 72 aa, synthetic construct
  REGION 1..72: note = Synthetic Polypeptide
ATVKFTYQGEEKQVDMSKIKHVRRWGQWIWFIYDEGGGARGRGYVSEKGAPEELLQMLGKQGSEQKLISEED

SEQ ID NO: 321 - protein, 72 aa, synthetic construct
  REGION 1..72: note = Synthetic Polypeptide
ATVKFTYQGREKQVDISKIKHVRRRGQYIWFGYDKGGGARGYGYVSEGDAPKELLQMLEKQESEQKLISEED

SEQ ID NO: 322 - protein, 69 aa, synthetic construct
  REGION 1..69: note = Synthetic Polypeptide
ATVKFTYQGEEKQVGISKIRHVRRWGQWIWFIYDEGGGAKGRGSVSERDAPKELLQMLEKARIRTKAYF

SEQ ID NO: 323 - protein, 72 aa, synthetic construct
  REGION 1..72: note = Synthetic Polypeptide
ATVKFTYQGGEKQVDISKIKHVWRWGQWVWFRYDEGGGARGYGRVSEKGAPKELLQMLGKQGSEQKLISEED

SEQ ID NO: 324 - protein, 72 aa, synthetic construct
  REGION 1..72: note = Synthetic Polypeptide
ATVKFTYHGEGKQVDISKIRYVRRWGRYIWFGYDEGGGARGYGYVSEKGAPEELLQMLGKQGSEQKLISEED

SEQ ID NO: 325 - protein, 69 aa, synthetic construct
  REGION 1..69: note = Synthetic Polypeptide
ATVKFTYRGEEKQVDISKIKHVRRWGQWIWFIYDEGGGAKGKGSVSEEDAPKELLQMLEKVGIRTKAYF

SEQ ID NO: 326 - protein, 72 aa, synthetic construct
  REGION 1..72: note = Synthetic Polypeptide
ATVKFTYRGGEKQVDISRVKYVWRRGQWIWFRYDGGGGAHGTGCVSEKNAPKELLQMLGRQGSEQKLISEED

SEQ ID NO: 327 - protein, 72 aa, synthetic construct
  REGION 1..72: note = Synthetic Polypeptide
ATVKFTYQGEEKQVDISKIKHVRRWGQWIWFIYDKGGGAKGRGGVSDKDAPKELLQMLEKQGSEQKLISEED

SEQ ID NO: 328 - protein, 72 aa, synthetic construct
  REGION 1..72: note = Synthetic Polypeptide
ATVRFTYRGEEKQVDISKIKHVRRWGQWIWFIYDEGGGAKGRGGVSKEDAPKELLRMLGKQGSEQKLISEED

SEQ ID NO: 329 - protein, 72 aa, synthetic construct
  REGION 1..72: note = Synthetic Polypeptide
ATVKFTYHGEERQVDISKIKHARRWGQWIWFIYDEGGGAKGRGGVSERNAPKELLQMLEGRGSEQKLISEED

SEQ ID NO: 330 - protein, 72 aa, synthetic construct
  REGION 1..72: note = Synthetic Polypeptide
ATVRFTYQGEEKQVDISKIKYVRRRGQYIWFGYDEGGGARGYGYVSGKGAPKELLQMLEEHGSEQKLISEED

SEQ ID NO: 331 - protein, 72 aa, synthetic construct
  REGION 1..72: note = Synthetic Polypeptide
ATVKFTYRGEGKQVDISKIKHVRRWGQWIWFIYDEGGGAKGWGGVSEKGAPKALLQMLEKQGSEQKLISEED

SEQ ID NO: 332 - protein, 72 aa, synthetic construct
  REGION 1..72: note = Synthetic Polypeptide
ATVEFTYRGEEKQVDISKIKHVRRWGQWIWFIYDEGGGAKGRGGVSERDAPKELLQLLEKRGSEQKLISEED

SEQ ID NO: 333 - protein, 72 aa, synthetic construct
  REGION 1..72: note = Synthetic Polypeptide
ATVKFTYQGKEKQVDISKIKYVRRRGQYIWFGYDKSGGARGYGYVSEKGAPKELLQMLEKQGSEQKLISEED

SEQ ID NO: 334 - protein, 72 aa, synthetic construct
  REGION 1..72: note = Synthetic Polypeptide
ATVKFTYRGGEKQVDIGKIKYVRRWGQYVWFGYDEGGGARGYGYVSEKDAPKELLQMLEKRGSEQKLISEED

SEQ ID NO: 335 - protein, 72 aa, synthetic construct
  REGION 1..72: note = Synthetic Polypeptide
ATVKFTHRGEEKQVDASKIKYVRRWGRHIWFGYDEGGGARGYGYVGEKDAPKELLQMLERQGSEQKLISEED

SEQ ID NO: 336 - protein, 72 aa, synthetic construct
  REGION 1..72: note = Synthetic Polypeptide
ATVKFTYQGEEKQVDISKIKHVRRWGQWIWFTYDEGGGAKGRGGVSEKDAPKELLQMLEKQGSEQKLISEED

SEQ ID NO: 337 - protein, 72 aa, synthetic construct
  REGION 1..72: note = Synthetic Polypeptide
TTVKFTYQGEEKQVDISKIKHVRRWGRWIWFTYDEGGGAKGRGGVSEKDAPKELLQMLEKRGSEQKLISEED

SEQ ID NO: 338 - protein, 72 aa, synthetic construct
  REGION 1..72: note = Synthetic Polypeptide
TTVKFTYQGEEKQVDISKIKHVRRWGQWIWFTYDEGGGAKGRGGVSEKDAPKELLRMLEKQGSEQKLISEED

SEQ ID NO: 339 - protein, 72 aa, synthetic construct
  REGION 1..72: note = Synthetic Polypeptide
ATVKFTYRGEEKQVAISKIKYVRRWGQHIWFGYDKGGGVRGYGYVGEKGAPRGLLQMLEKQGSEQKLISEED

SEQ ID NO: 340 - protein, 72 aa, synthetic construct
  REGION 1..72: note = Synthetic Polypeptide
ATVRFTYQGEEKQVDINRIKHVRRWGQWIWFIYDEGGGAKGRGGVSGKDAPKELLQMLEKQGSEQKLISEED

SEQ ID NO: 341 - protein, 72 aa, synthetic construct
  REGION 1..72: note = Synthetic Polypeptide
ATVKYTYRGEEKQVGISRIKSVHRIGQWIKFWYDEGSGAYGRGYVSEKDAPRELLQMLGKRGSEQKLISEED

SEQ ID NO: 342 - protein, 72 aa, synthetic construct; identical to SEQ ID NO: 28
  REGION 1..72: note = Synthetic Polypeptide

SEQ ID NO: 343 - protein, 69 aa, synthetic construct
  REGION 1..69: note = Synthetic Polypeptide
ATVKFTYQGEEKQVDISKIKYVRRWGQYIWFGYDAGGGVRGYGYVSEKDAPKGLLQMLEKARIRTKAYF

SEQ ID NO: 344 - protein, 72 aa, synthetic construct
  REGION 1..72: note = Synthetic Polypeptide
ATVKFTYQGEEKQVNISKIKHVRRWGQWIWFVYDEGGGAKGRGGVSEKDAPKELLQMLEKQGSEQKLISEED

SEQ ID NO: 345 - protein, 72 aa, synthetic construct
  REGION 1..72: note = Synthetic Polypeptide
ATVKFTYRGEEKQVDISEIRYVWRRGQWIWFRYDEGGGAHGIGHVSEKGAPKELLQTLERQGSEQKLISEED

SEQ ID NO: 346 - protein, 72 aa, synthetic construct
  REGION 1..72: note = Synthetic Polypeptide
ATVKFTYRGEEKRVDISKIKHVRRWGQWIWFIYDEGGGAHGRGRVSEKDAPKELLQMLEKQGSEQKLISEED

SEQ ID NO: 347 - protein, 72 aa, synthetic construct
  REGION 1..72: note = Synthetic Polypeptide
ATVKFTYQGGKKQMDISKLKYVRRWGRYIWFGYDEGGGARGYGYVSGKDAPRELLQMLEKQGSEQKLISEED

SEQ ID NO: 348 - protein, 72 aa, synthetic construct
  REGION 1..72: note = Synthetic Polypeptide
ATVKFTYRGEEKQVDISKIKYVRRWGQYVWFGYDEGGGAKGRGGVSKKDAPKELLQMLERQGSEQKLISEED

SEQ ID NO: 349 - protein, 72 aa, synthetic construct
  REGION 1..72: note = Synthetic Polypeptide
ATVKFTYQGEEKQVDISKIKYVRRWGQYIWFGYDEGGGARGRGYVSGKDAPKELLQMLEKQGSEQKLISEED

SEQ ID NO: 350 - protein, 72 aa, synthetic construct
  REGION 1..72: note = Synthetic Polypeptide
ATVKFTYQGGEKQVGISRIKYVRRRGQYIWFGYDKGGGARGYGYVSEKDAPKELLQMLEKQGSEQKLISEED

SEQ ID NO: 351 - protein, 72 aa, synthetic construct
  REGION 1..72: note = Synthetic Polypeptide
ATVKFTYQGEEKQVDISKIKYVRRWGQYIWFGYDAGGGARGYGYVSEKDAPKGLLQMLEKRGSEQKLISEED

SEQ ID NO: 352 - protein, 72 aa, synthetic construct
  REGION 1..72: note = Synthetic Polypeptide
ATVKFTYQGEEKQVDISRIKYVRRWGQYIWFGYGEGGGARGYGYVSEKDAPEGLLQMLGKRGSEQKLISEED

SEQ ID NO: 353 - protein, 72 aa, synthetic construct
  REGION 1..72: note = Synthetic Polypeptide
AAVKFTYQGEERQVDISKIKHVRRWGQWIWFIYDEGGGAKGRGGVSEKDAPKELLQMLERQGSEQKLISEED

SEQ ID NO: 354 - protein, 72 aa, synthetic construct
  REGION 1..72: note = Synthetic Polypeptide
ATVKFTYRGEEKQADISKIKYVRRWGQYVWFGYDEGGGVRGYGYVSEKDAPKELLQMLEKQGSEQKLISEED

SEQ ID NO: 355 - protein, 72 aa, synthetic construct
  REGION 1..72: note = Synthetic Polypeptide
ATVKFTYQGEEKQVDISKIKHVRRWGQWIWFVYDKGGGAKGRGGVSEKNAPKELLQMLERQGSEQKLISEED

SEQ ID NO: 356 - protein, 72 aa, synthetic construct
  REGION 1..72: note = Synthetic Polypeptide
AIVKFTYHGEEKQVDISKIKYVRRWGQYIWFGYDEGGGARGYGYVSERGAPKELLQMLEKRGSEQKLISEED

SEQ ID NO: 357 - protein, 72 aa, synthetic construct
  REGION 1..72: note = Synthetic Polypeptide
ATVKFTYRGGEKQVDISKIKYVRRRGQYIWFGYDEGGGARGYGYVSERDAPKELLQMLEKQGSEQKLISEED

SEQ ID NO: 358 - protein, 72 aa, synthetic construct
  REGION 1..72: note = Synthetic Polypeptide
ATVKFTYQGEEKQVDISKIKHVRRWGQWIWFIYDKGGGAKGRGGVSEKNAPKELLQMLEKQGSEQKLISEED

SEQ ID NO: 359 - protein, 72 aa, synthetic construct; identical to SEQ ID NO: 30
  REGION 1..72: note = Synthetic Polypeptide

SEQ ID NO: 360 - protein, 72 aa, synthetic construct
  REGION 1..72: note = Synthetic Polypeptide
ATVKFTYRGEGKQVDISKIKYVRRWGQYVWFGYDEGGGARGYGYVSEKDAPKELLQMLEKQGSEQKLISEED

SEQ ID NO: 361 - protein, 72 aa, synthetic construct
  REGION 1..72: note = Synthetic Polypeptide
ATVKFTYRGGEKQVDISRIKYVWRRGQWIWFRYDGGGGAHGTGCVSEKNAPKELLQMLGRQGSEQKLISEED

SEQ ID NO: 362 - protein, 72 aa, synthetic construct
  REGION 1..72: note = Synthetic Polypeptide
ATVEFTYRGEEKQVDVSKIKYAWRWGRWIWFRYDEGGSAHGIGYVSEKDAPKELLQMLEKQGSEQKLISEED

SEQ ID NO: 363 - protein, 72 aa, synthetic construct
  REGION 1..72: note = Synthetic Polypeptide
ATVKFTYQGEEKQVGVSRITYVRRRGQYIWFGYDKGGGARGYGYVSEKDAPKELLQMLEKQGSEQKLISEED

SEQ ID NO: 364 - protein, 72 aa, synthetic construct
  REGION 1..72: note = Synthetic Polypeptide
ATVKFTYRGEEKQVDISKIKYVRRWGQYIWFGYDGGGGANGRGGVSERGAPKELLQMLGKQGSEQKLISEED

SEQ ID NO: 365 - protein, 72 aa, synthetic construct
  REGION 1..72: note = Synthetic Polypeptide
ATVEFTYQGEEKQVDIGKIKYVRRWGQYIWFGYDEGGGARGYGYVSRKGAPKELLQMLEKRGSEQKLISEED

SEQ ID NO: 366 - protein, 72 aa, synthetic construct
  REGION 1..72: note = Synthetic Polypeptide
AIVRFTYRGEEKRVDISEIKYVRRWGQYIWFGYDKGGGARGHGYVSEKDAPKELLQMLEEQGSEQKLISEED

SEQ ID NO: 367 - protein, 72 aa, synthetic construct
  REGION 1..72: note = Synthetic Polypeptide
ATVKFTYRGEEKQVDISKIKHARRWGQYIWFGYDEGGGARGYGYVSEKDAPKELLRMLEKRGSEQKLISEED

SEQ ID NO: 368 - protein, 60 aa, synthetic construct
  REGION 1..60: note = Synthetic Polypeptide
ATVKFTYQGEEKQVDISRIKHVRRRGQYIWFGYDEGGGARGYGYVSEKDAPKELLRMLEK

SEQ ID NO: 369 - protein, 72 aa, synthetic construct
  REGION 1..72: note = Synthetic Polypeptide
ATVKFTYRGEEKQVDISKIKYVRRWGQYIWFGYDEGGGARGHGRVSEKDAPRGLLQMLEKQGSEQKLISEED

SEQ ID NO: 370 - protein, 72 aa, synthetic construct
  REGION 1..72: note = Synthetic Polypeptide
AIVKFTHHGEEKQVDISKIKYVRRWGQYIWFGYDEGGGARGYGYVSERDAPKELLQMLEKRGSEQKLISEED

SEQ ID NO: 371 - protein, 72 aa, synthetic construct
  REGION 1..72: note = Synthetic Polypeptide
TTVKFTYQGEEKQVGISRIKYVRRRGQYIWFGYDKGGGARGYGYVSGKDAPRELLQMLEKQGSEQKLISEED

SEQ ID NO: 372 - protein, 72 aa, synthetic construct
  REGION 1..72: note = Synthetic Polypeptide
ATVRFTYHGEEKQVDISKIKYARRWGQYIWFGYDEGGGARGYGHVSGEDAPKELLQMLEKPGSEQKLISEED

SEQ ID NO: 373 - protein, 72 aa, synthetic construct
  REGION 1..72: note = Synthetic Polypeptide
ATVKFTYQGEEKQVDISKIKYVRRWGRYIWFGYDGGGGARGYGHVSEKDAPKELLQMLGGRGSEQKLISEED

SEQ ID NO: 374 - protein, 72 aa, synthetic construct
  REGION 1..72: note = Synthetic Polypeptide
ATVKFTYQGGEKQVDISKVRHVRRWGRWIWFGYDEGGGAHGRGRVSGKDAPKELLQMLEKQGSEQKLISEED

SEQ ID NO: 375 - protein, 72 aa, synthetic construct
  REGION 1..72: note = Synthetic Polypeptide
ATVKFTYRGEEKQVDISKIKRVRRWGQWIWFIYDEAGGAHGRGRVSERGAPKELLQMLEKQGSEQKLISEED

SEQ ID NO: 376 - protein, 72 aa, synthetic construct
  REGION 1..72: note = Synthetic Polypeptide
ATVEFTYQGEEKQVDISKIKHVRRRGQYIWFGYDKGGGARGYGYVSEKDAPKELLQMLEKQGSEQKLISEED

SEQ ID NO: 377 - protein, 72 aa, synthetic construct
  REGION 1..72: note = Synthetic Polypeptide
ATVKFTYRGEEKQVDISKIKHVRRWGQWIWFIYDEGGGAKGWGGVSEKDAPRGLLQMLERQGSEQKLISEED

SEQ ID NO: 378 - protein, 72 aa, synthetic construct
  REGION 1..72: note = Synthetic Polypeptide
ATVKFTYQGEEKQVDISKIKYVRRWGQWIWFRYDGGGGAHGIGHVSEKDAPKELLQMLGKQGSEQKLISEED

SEQ ID NO: 379 - protein, 72 aa, synthetic construct
  REGION 1..72: note = Synthetic Polypeptide
ATVKFTYHGEEKQVDISKIKYVRRWGQYIWFGYDEGGGARGYGYVSGKGAPEELLQMLEKQGSEQKLISEED

SEQ ID NO: 380 - protein, 72 aa, synthetic construct
  REGION 1..72: note = Synthetic Polypeptide
ATVKFTYQGEGKQVDISKIKHVRRWGQWIWFIYDEGGGARGYGYVSGKDAPKKLLRMLEGRGSEQKLISEED

SEQ ID NO: 381 - protein, 72 aa, synthetic construct
  REGION 1..72: note = Synthetic Polypeptide
AAVEFTYQGEEKQVDISKIKHVRRWGQWIWFIYDEAGGAHGRGRVSERGAPKELLQMLERQGSEQKLISEED

SEQ ID NO: 382 - protein, 72 aa, synthetic construct
  REGION 1..72: note = Synthetic Polypeptide
ATVKFTYRGGEKQVDISKIKYVRRRGQYIWFGYDEGGGARGYGYVSERDAPRELLQMLEKQGSEQKLISEED

SEQ ID NO: 383 - protein, 72 aa, synthetic construct
  REGION 1..72: note = Synthetic Polypeptide
ATVKFTYRGEEKQVDISKIKYVRRRGQYIWFGYDEGGGARGYGYVSEKDAPKELLQMLGKQGSEQKLISEED

SEQ ID NO: 384 - protein, 72 aa, synthetic construct
  REGION 1..72: note = Synthetic Polypeptide
ATVKFTYRGEEKRVDTSKIKHVRRWGQWIWFTYDEGGGAKGRGGVSEKDAPKELLQMLEKQGSEQKLISEED

SEQ ID NO: 385 - protein, 72 aa, synthetic construct
  REGION 1..72: note = Synthetic Polypeptide
ATVKFTYRGEEKQVDISKIKYVWRWGQWIWFRYDEGGGAHGIGHVSEKSAPKELLQTLGRQGSEQKLISEED

SEQ ID NO: 386 - protein, 72 aa, synthetic construct
  REGION 1..72: note = Synthetic Polypeptide
ATVKSTYQGEEKQVDISKIKHVRRWGRWIWFIYDEGGGAKGWGGVSGRDAPKELLQMLEKQGSEQKLISEED

SEQ ID NO: 387 - protein, 72 aa, synthetic construct
  REGION 1..72: note = Synthetic Polypeptide
AIVKFTYQGEERQVDISKIKYVRRWGQYIWFGYDEGGGAHGRGRVSGKDAPKELLQMLEKQGSEQKLISEED

SEQ ID NO: 388 - protein, 72 aa, synthetic construct
  REGION 1..72: note = Synthetic Polypeptide
ATVKFTYHGEERQVDISKIKYVRRWGQYIWFGYGGGGGARGYGYVSEKDAPKELLQMLEKQGSEQKLISEED

SEQ ID NO: 389 - protein, 72 aa, synthetic construct
  REGION 1..72: note = Synthetic Polypeptide
ATVKFTYQGEEKQVDISKIKYVRRWGQYIWFGYDGGGGARGRGYVSEKDAPKELLQMLEKQGSEQKLISEED

SEQ ID NO: 390 - protein, 72 aa, synthetic construct
  REGION 1..72: note = Synthetic Polypeptide
VAVKFTYQGEEKRVDISKIKYVRRRGQYIWFGYGEGGGARGYGYVSEKDAPKELLQMLAKRGSEQKLISEED

SEQ ID NO: 391 - protein, 72 aa, synthetic construct
  REGION 1..72: note = Synthetic Polypeptide
ATVKFTYQGEEKQVDISKIKHVRRWGQWIWFIYDGGGGAKGRGGVSEKDAPKELLQMLEKQGSEQKLISEED

SEQ ID NO: 392 - protein, 72 aa, synthetic construct
  REGION 1..72: note = Synthetic Polypeptide
ATVKFTYRGEEKQVDASRIKYVRRWGQYIWFGYDEGGGARGYGYVSGRDAPKELLQMLEKQGSEQKLISEED

SEQ ID NO: 393 - protein, 72 aa, synthetic construct
  REGION 1..72: note = Synthetic Polypeptide
ATVKFTYQGEEKQVDISKIRYARRRGQYIWFGYGEGGGARGYGYVSDKDAPKELLRMLEKQGSEQKLISEED

SEQ ID NO: 394 - protein, 73 aa, synthetic construct
  REGION 1..73: note = Synthetic Polypeptide
ATVKFTYRGEEKQVGISRIRSVHRIGQWIKFWYDEGSGACGRGYVSEKGAPKELLQMLGKRGSEQKLISEEDL

SEQ ID NO: 395 - protein, 73 aa, synthetic construct
  REGION 1..73: note = Synthetic Polypeptide
ATVKFTYRGKEKRVGVSRIKSVRRIGQWIKFWYDEGSGAYGRGYVSEKDAPKELLQMLEKRGSEQKLISEEDL

SEQ ID NO: 396 - protein, 73 aa, synthetic construct
  REGION 1..73: note = Synthetic Polypeptide
ATVKFTYRGEEKQVGINRIKSVHRIGQWIKFWYDEGSGAYGRGYVSGKDAPKELLRMLEKRGSEQKLISEEDL

SEQ ID NO: 397 - protein, 73 aa, synthetic construct
  REGION 1..73: note = Synthetic Polypeptide
ATVKFTYQGEEKQVDISKIKIVYRWGQRISFIYDEGGGARGYGRVSEKDAPKELLQMLEKQGSEQKLISEEDL

SEQ ID NO: 398 - protein, 73 aa, synthetic construct
  REGION 1..73: note = Synthetic Polypeptide
ATVKFTYQGEEKQVDISKIKHVRRWGQWIWFIYDEGGGAKGWGGVSEKDAPKELLQMLEKQGSEQKLISEEDL

SEQ ID NO: 399 - protein, 73 aa, synthetic construct
  REGION 1..73: note = Synthetic Polypeptide
ATVKFTYQGEEKQVDISKIKYVRRWGQYIWFGYDEGGGARGYGYVSEKDAPKELLQMLEKQGSEQKLISEEDL

SEQ ID NO: 400 - protein, 73 aa, synthetic construct
  REGION 1..73: note = Synthetic Polypeptide
ATVKFTYQGEEKQVDISKIKRVYRYGQWIWFRYDEGGGAYGGGWVSEKDAPKELLQMLEKQGSEQKLISEEDL

SEQ ID NO: 401 - protein, 69 aa, synthetic construct
  REGION 1..69: note = Synthetic Polypeptide
ATVKFTYQGEEKQVDISKIKSVSRWGQAIIFRYDEGGGAKGKGSVSEKDAPKELLQMLEKARIRTKAYF

SEQ ID NO: 402 - protein, 61 aa, synthetic construct
  REGION 1..61: note = Synthetic Polypeptide
ATVKFTYRGEEKQVGISRIKSVRRIGQWIKFWYDEGSGAYGRGYVSEKDAPKELLQMLEKR

SEQ ID NO: 403 - protein, 61 aa, synthetic construct
  REGION 1..61: note = Synthetic Polypeptide
ATVKFTYRGEEKQVGISRIKSVRRIGQWIKFWYDEGSGAYGRGYVSGKDAPKELLQMLEKR

SEQ ID NO: 404 - protein, 61 aa, synthetic construct
  REGION 1..61: note = Synthetic Polypeptide
ATVKFTYRGGEKQVGISRIKSVRRIGQWIKFWYDEGSGAYGRGYVSEKDAPKELLQMLEKR

SEQ ID NO: 405 - protein, 61 aa, synthetic construct
  REGION 1..61: note = Synthetic Polypeptide
ATVKFTYRGEEKQVGISRIKSVHRIGRWIKFWYDEGSGAYGRGYVSEKDAPKELLQMLEKR

SEQ ID NO: 406 - protein, 61 aa, synthetic construct
  REGION 1..61: note = Synthetic Polypeptide
ATVKFTYRGEEKQVGISRIKSVHRIGQWIKFWYDEGSGAYGRGYVSKKDAPKELLQMLEKR

SEQ ID NO: 407 - protein, 7 aa, synthetic construct
  REGION 1..7: note = Synthetic Polypeptide
KKVWRVG

SEQ ID NO: 408 - protein, 7 aa, synthetic construct
  REGION 1..7: note = Synthetic Polypeptide
QMISFTY

SEQ ID NO: 409 - protein, 7 aa, synthetic construct
  REGION 1..7: note = Synthetic Polypeptide
ATGRGAV

SEQ ID NO: 410 - protein, 10 aa, synthetic construct
  REGION 1..10: note = Synthetic Polypeptide
QVGVSRVKSV

SEQ ID NO: 411 - protein, 10 aa, synthetic construct
  REGION 1..10: note = Synthetic Polypeptide
QVDISKIKKV

SEQ ID NO: 412 - protein, 7 aa, synthetic construct
  REGION 1..7: note = Synthetic Polypeptide
  VARIANT 2: note = X can be any naturally occurring amino acid
  VARIANT 2..6: note = Xs cannot simultaneously be KWV, sequentially
  VARIANT 4: note = X can be any naturally occurring amino acid
  VARIANT 6: note = X can be any naturally occurring amino acid
KXVXRXG

SEQ ID NO: 413 - protein, 7 aa, synthetic construct
  REGION 1..7: note = Synthetic Polypeptide
  VARIANT 2: note = X can be any naturally occurring amino acid
  VARIANT 2..6: note = Xs cannot simultaneously be MST, sequentially
  VARIANT 4: note = X can be any naturally occurring amino acid
  VARIANT 6: note = X can be any naturally occurring amino acid
QXIXFXY

SEQ ID NO: 414 - protein, 7 aa, synthetic construct
  REGION 1..7: note = Synthetic Polypeptide
  VARIANT 2: note = X can be any naturally occurring amino acid
  VARIANT 2..6: note = Xs cannot simultaneously be TRA, sequentially
  VARIANT 4: note = X can be any naturally occurring amino acid
  VARIANT 6: note = X can be any naturally occurring amino acid
AXGXGXV

SEQ ID NO: 415 - protein, 59 aa, synthetic construct
  REGION 1..59: note = Synthetic Polypeptide
ATVKFTYQGEEKQVDISKIKSVHRVGQWIKFWYDGGGGAYGRGYVSEKDAPKELLQMLE

SEQ ID NO: 416 - protein, 61 aa, synthetic construct
  REGION 1..61: note = Synthetic Polypeptide
ATVKFTYRGEEKQVDISKIKYVRRWGQYIWFGYDEGGGARGYGYVSETDAPELLLQMLEKQ

SEQ ID NO: 417 - protein, 61 aa, synthetic construct
  REGION 1..61: note = Synthetic Polypeptide
ATVKFTYRGEGKQVGISRIKSVRRIGQWIKFWYDEGSGAYGRGYVSGKDAPKELLQMLEKR

SEQ ID NO: 418 - protein, 61 aa, synthetic construct
  REGION 1..61: note = Synthetic Polypeptide
ATVKFTYRGEEKQVGISRIKSVRRIGRWIKLWYDEGSGAYGRGYVSGKDAPKELLQMLEKR

(19) United States
(12) Patent Application Publication
(10) Pub. No.: US 2024/0101620 A1
(43) Pub. Date: Mar. 28, 2024

(54) GLYCAN-BINDING PROTEINS AND RELATED COMPOSITIONS AND METHODS

(71) Applicant: Massachusetts Institute of Technology, Cambridge, MA (US)

(72) Inventors: Barbara Imperiali, Cambridge, MA (US); Cristina Zamora, Medford, MA (US); Elizabeth Ward, Somerville, MA (US)

(73) Assignee: Massachusetts Institute of Technology, Cambridge, MA (US)

(21) Appl. No.: 18/160,144

(22) Filed: Jan. 26, 2023

Related U.S. Application Data
(63) Continuation of application No. 16/818,827, filed on Mar. 13, 2020, now Pat. No. 11,597,751.
(60) Provisional application No. 62/848,891, filed on May 16, 2019.

(51) Int. Cl. C07K 14/47

(52) U.S. Cl. CPC C07K 14/47

(57) ABSTRACT

Glycan-binding proteins, and compositions thereof, are generally described, including methods of making and using such proteins. The proteins may include scaffolds based on easily evolvable DNA-binding proteins, with binding sites able to specifically bind to mono- or disaccharides, such as monosaccharide-binding determinants, disaccharide-binding determinants, more complex carbohydrates, etc. In certain aspects, a protein may be generated starting from a small DNA-binding protein, such as Sso7d. Such glycan-binding proteins may have numerous applications, including in enzyme-linked immunosorbent assays (ELISAs), glycan characterization, cell selection, flow cytometry, histology, imaging, arrays, affinity purification, enzyme-linked visualization, binding to a target for pharmaceutical purposes, etc.

### RELATED APPLICATIONS

This application is a continuation of U.S. patent application Ser. No. 16/818,827, filed on Mar. 13, 2020, and entitled “Glycan-Binding Proteins and Related Compositions and Methods,” which claims priority to U.S. Provisional Patent Application Ser. No. 62/848,891, filed on May 16, 2019, and entitled “Glycan-Binding Proteins and Related Compositions and Methods,” each of which is hereby incorporated by reference in its entirety.

### GOVERNMENT SPONSORSHIP

This invention was made with government support under AI130776 awarded by the National Institutes of Health. The government has certain rights in the invention.

### REFERENCE TO AN ELECTRONIC SEQUENCE LISTING

The contents of the electronic sequence listing (M092570757US02-SEQ-LBS.xml; Size: 558,992 bytes; and Date of Creation: Jan. 26, 2023) is herein incorporated by reference in its entirety.

### TECHNICAL FIELD

Glycan-binding proteins and related compositions and methods are generally described.

### SUMMARY

Glycan-binding proteins, and compositions thereof, are generally described. Inventive methods of making and using the glycan-binding proteins are also described. The subject matter of the present invention involves, in some cases, interrelated products, alternative solutions to a particular problem, and/or a plurality of different uses of one or more systems and/or articles.

Certain aspects are related to compositions. In one aspect, a composition comprises a protein having at least 55% homology to the following sequence:

(SEQ ID NO: 3)
ATVKFTYQGEEKQVDISKIK(s1)(s2)DEGGG(s3)SEKDAPKELLQML
EKQ

wherein (s1) consists of 7 amino acid residues and is not KKVWRVG (SEQ ID NO: 407), (s2) consists of 7 amino acid residues and is not QMISFTY (SEQ ID NO: 408), (s3) consists of 7 amino acid residues and is not ATGRGAV (SEQ ID NO: 409). In some embodiments, the protein specifically binds to a monosaccharide or disaccharide-binding determinant.

In another aspect, a composition comprises a protein having at least 55% homology to the following sequence:

(SEQ ID NO: 4)
ATVKFTYQGEEKQVDISKIKKX¹VX²RX³GQX⁴IX⁵FX⁶YDEGGGAX⁷GX⁸G
X⁹VSEKDAPKELLQMLEKQ,

wherein each of X¹, X², X³, X⁴, X⁵, X⁶, X⁷, X⁸, and X⁹ is independently an amino acid residue, with the proviso that X¹, X², X³, X⁴, X⁵, X⁶, X⁷, X⁸, and X⁹ cannot simultaneously be K, W, V, M, S, T, T, R, and A, respectively. In some cases, the protein specifically binds to a monosaccharide or disaccharide-binding determinant.

In another aspect, a composition comprises a protein having 55-99% homology to the following sequence:

(SEQ ID NO: 1)
ATVKFTYQGEEKQVDISKIKKVWRVGQMISFTYDEGGGATGRGAVSEKD
APKELLQMLEKQ,

wherein the protein specifically binds to a monosaccharide or disaccharide-binding determinant.

In yet another aspect, a composition comprises a first glycan-binding portion and a second glycan-binding portion. In some cases, each of the first glycan-binding portion and the second glycan-binding portion independently has at least 55% homology to Sso7d.

In addition, certain aspects are related to methods. For example, in one aspect, a method of producing a glycan-binding protein comprises providing a protein scaffold, wherein the protein scaffold comprises Sso7d, generating one or more variants of the protein scaffold, determining binding and/or binding selectivity of the one or more variants to a monosaccharide or disaccharide-binding determinant, selecting a variant exhibiting increased binding and/or binding selectivity to the monosaccharide or disaccharide-binding determinant from the one or more variants, and repeating the generating, determining and selecting steps, using the variant exhibiting increased binding and/or binding selectivity to the monosaccharide or disaccharide-binding determinant in each repeat.

In another aspect, a method of producing a glycan-binding protein comprises providing a protein scaffold, wherein the protein scaffold has no more than 200 amino acid residues, with a binding face area of less than or equal to 6 square nanometers (nm²), generating one or more variants of the protein scaffold, determining binding and/or binding selectivity of the one or more variants to a monosaccharide or disaccharide-binding determinant, selecting a variant exhibiting increased binding and/or binding selectivity to the monosaccharide or disaccharide-binding determinant from the one or more variants, and repeating the generating, determining and selecting steps, using the variant exhibiting increased binding and/or binding selectivity to the monosaccharide or disaccharide-binding determinant in each repeat.

A large variety of proteins are described herein. For example, in one set of embodiments, the protein is selected from Sequence List 1. In another set of embodiments, the protein is selected from Sequence List 2.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates a flowchart of methods of generating a glycan-binding protein, in some embodiments.

FIG. 2A illustrates the structure of Galβ1-3GalNAcα (TF or Thomsen-Friedenrich antigen).

FIG. 2B illustrates the structure of Galα1-3GalNAcα, with arrows towards various points of differentiation from the TF antigen.

FIG. 2C illustrates the structure of GalNAcα1-3GalNAcα, with arrows towards various points of differentiation from the TF antigen.

FIG. 2D illustrates, in accordance with certain embodiments, the percent binding of the three compounds of FIGS. 2A-2C and PAA-FITC (the control) for five different glycan-binding proteins.

FIG. 2E illustrates biolayer interferometry traces of a glycan-binding protein in accordance with some embodiments.

FIG. 3A illustrates the structure of Neu5Ac.

FIG. 3B illustrates the structure of Neu5Gc.

FIGS. 3C-3E illustrate flow cytometry results for sialic acid-PAA-FITC (FIG. 3C), NeuN5Gc-PAA-FITC (FIG. 3D), and PAA-FITC (FIG. 3E) for a glycan-binding protein, in accordance with certain embodiments.

FIG. 4 illustrates, in accordance with certain embodiments, a histogram of the percent identity of the glycan-binding proteins in Sequence List 2 with rcSso7d.

FIG. 5 illustrates functionalization and uses of the glycan-binding proteins, in accordance with some embodiments as described herein.

FIG. 6 illustrates conjugation of glycan-binding proteins, in accordance with various embodiments described herein.

FIG. 7 illustrates a yeast-surface display of a glycan-binding protein binding a sugar-binding determinant, in accordance with certain embodiments.

FIG. 8A illustrates the dimensions of an example disaccharide (i.e., TF antigen).

FIG. 8B illustrates the dimensions of an example monosaccharide (i.e., NeuN5Ac).

FIGS. 9A-9F illustrate disaccharides (or disaccharide motifs within trisaccharides) bound by glycan-binding proteins, in accordance with some embodiments described herein.

FIG. 10A illustrates median fluorescence intensity of a binding study of an embodiment described herein tested against various glycans.

FIG. 10B illustrates binding specificity of an embodiment described herein tested against various glycans.

FIG. 10C illustrates structures of all glycans tested for binding.

FIG. 10D illustrates biolayer interferometry traces of an embodiment described herein with apparent Kd values calculated.

FIG. 11A illustrates the binding specificity of embodiments described herein.

FIG. 11B illustrates biolayer interferometry traces of embodiments described herein with apparent Kd values calculated.

### BRIEF DESCRIPTION OF THE SEQUENCES

SEQ ID NO: 1 is a reduced-charge variant of Sso7d (rcSso7d), having a sequence:

ATVKFTYQGEEKQVDISKIKKVWRVGQMISFTYDEGGGATGRGAVSEKDA
PKELLQMLEKQ.

SEQ ID NO: 2 is Sso7d, a protein from S. solfataricus having a sequence:

ATVKFKYKGEEKQVDISKIKKVWRVGKMISFTYDEGGGKTGRGAVSEKDA
PKELLQMLEKQK.

SEQ ID NO: 3 is ATVKFTYQGEEKQVDISKIK(s1)(s2)DEGGG(s3) SEKDAPKELLQMLEKQ, where (s1) consists of 7 amino acid residues and is not KKVWRVG (SEQ ID NO: 407), (s2) consists of 7 amino acid residues and is not QMISFTY (SEQ ID NO: 408), and (s3) consists of 7 amino acid residues and is not ATGRGAV (SEQ ID NO: 409).

SEQ ID NO: 4 is the following amino acid sequence: ATVKFTYQGEEKQVDISKIKKX¹VX²RX³GQX⁴IX⁵FX⁶YDEGGGAX⁷GX⁸GX⁹VSE KDAPKELLQMLEKQ, where each of X¹, X², X³, X⁴, X⁵, X⁶, X⁷, X⁸, and X⁹ is independently an amino acid residue, with the proviso that X¹, X², X³, X⁴, X⁵, X⁶, X⁷, X⁸, and X⁹ cannot simultaneously be K, W, V, M, S, T, T, R, and A, respectively.

SEQ ID NO: 5 is M11.1, an artificial protein having the following sequence:

ATVKFTYQGEEKQVDISKIKWVIRWGQHIAFKYDEGGGAAGYGWVSEKDA
PKELLQMLEKQ.

SEQ ID NO: 6 is M11.2, an artificial protein having the following sequence:

ATVKFTYQGEEKQVDISKIKWVNRWGQRIYFKYDEGGGAAGYGWVSEKDA
PKELLQMLEKQ.

SEQ ID NO: 7 is M11.1.2, an artificial protein having the following sequence:

ATVKYTYRGEEKRVDISKIKWVNRWGQHLAFKYDKGGGAAGYGWVSEKDAP
KELLQMLEKR.

SEQ ID NO: 8 is M11.1.3, an artificial protein having the following sequence:

ATVKSTYRGEEKQVDISKIKWVIRWGQHLAFKYDEGGGAAGYGWVSEKDAP
KELLQMLEKQ.

SEQ ID NO: 9 is M11.1.5, an artificial protein having the following sequence:

ATVKFTYRGEEKQVDISKIKWVNRWGQHLAFKYDVGGGAAGYGWMSEKDAP
KELLQMLEKR.

SEQ ID NO: 10 is M18.1, an artificial protein having the following sequence:

ATVKFTYQGEEKQVDISKIKWVIRLGRTIMFKYDEGGGANGYGKVSEKDAP
KELLQMLEKQ.

SEQ ID NO: 11 is M18.2, an artificial protein having the following sequence:

ATVKFTYQGEEKQVDISKIKWVVRLGQVIMFKYDEGGGANGYGKVSEKDAP
KELLQMLEKQ.

SEQ ID NO: 12 is M18.2.2, an artificial protein having the following sequence:

ATVKFTYRGEEKQVDISKIKWVVRLGQVIMFKYGEGGGSNGYGRVSEKDA
PKELRQMLEKR.

SEQ ID NO: 13 is M18.2.5, an artificial protein having the following sequence:

ATVKFTYRGEEKQVDISKIKWVVRLGQVIMFKYDEGGGASGYGRVSEKDA
PKELLQMLEK.

### DETAILED DESCRIPTION

Glycan-binding proteins, and compositions thereof, are generally described, including methods of making and using such proteins. The proteins may include scaffolds based on easily evolvable DNA-binding proteins, with binding sites able to specifically bind to mono- or disaccharides, such as monosaccharide-binding determinants, disaccharide-binding determinants, in more complex carbohydrates, etc. In certain aspects, a protein may be generated starting from a small DNA-binding protein, such as Sso7d. Such glycan-binding proteins may have numerous applications, including in enzyme-linked immunosorbent assays (ELISAs), glycan characterization, cell selection, flow cytometry, histology, imaging, arrays, affinity purification, enzyme-linked visualization, binding to a target for pharmaceutical purposes, etc.

Certain aspects of the invention are generally directed to proteins able to bind to glycans, for example, via specific binding. Glycans are generally sugars or carbohydrates, alone or conjugated to other entities, such as proteins, lipids, small molecules, or the like. The glycans may include any number of saccharide units, including monosaccharides, disaccharides, and larger polysaccharides. Glycans can be homo- or heteropolymers of monosaccharide residues, and can be linear or branched. The glycan may comprise only saccharide units, or other non-saccharide units as well, for example, as in glycoproteins, glycolipids, glyconucleic acids, proteoglycans, etc.

In some cases, glycan-binding proteins such as those discussed herein may be relatively small or low-molecular weight, and can accordingly bind to small glycan-binding determinants, e.g., monosaccharides or disaccharides within an overall glycan structure, e.g., via specific binding. Such glycan-binding determinants that the protein can bind may be a single monosaccharide or disaccharide, or in some cases, the glycan-binding determinant may be part of a larger structure, e.g., such as those noted above.

In contrast, other carbohydrate-binding proteins known to the art are typically significantly larger, and are unable to specifically bind to or recognize single monosaccharide or disaccharide-binding determinants. Glycan-binding proteins such as these may be useful in a variety of immunological, therapeutic, diagnostic, or technological roles such as those discussed herein.

In addition, certain embodiments of the invention are generally directed to systems and methods for making such glycan-binding proteins. In some cases, a DNA-binding protein may be used as a protein scaffold and engineered, e.g., using directed evolution, to produce a glycan-binding protein. In some cases, e.g., after multiple generations, proteins with high specificities of binding to glycans may be developed.

In some cases, the protein scaffold may be one that is readily evolvable. The protein scaffold may also, in certain embodiments, have a binding site (e.g., a binding pocket) that has dimensions compatible with monosaccharide and/or disaccharide binding, and/or have a binding site (e.g., a binding pocket) that has dimensions similar to those of any monosaccharide or disaccharide motif of interest within a glycan.

In addition, in certain embodiments, the protein scaffold may be devoid of disulfides. In some cases, the protein scaffold may be stable to a wide range of temperatures and/or pH values. In addition, such protein scaffolds may be one that can be readily functionalized chemically or conjugated to other entities, for example, to generate clustered or branched assemblies. For example, in one set of embodiments, two such protein scaffolds may be linked together.

As one non-limiting example, in some embodiments, Sso7d (or a reduced-charge variant thereof) can be used as a protein scaffold. Native or wild-type Sso7d arises from Sulfolobus solfataricus, where it binds DNA and does not ordinarily bind glycans. However, the Sso7d scaffold can be used to develop glycan-binding proteins, as discussed herein. For instance, in some embodiments, the Sso7d protein scaffold is mutated, for example, by error-prone PCR, to generate variants. These variants are then, in some cases, analyzed to determine binding efficiency to a target glycan, for instance, using Yeast-Surface Display (YSD) selections with magnetic bead-immobilized glycans. The variant or variants with the best binding and/or binding selectivity to the target glycan (e.g., a specific monosaccharide or disaccharide-binding determinant) are then selected, and the process is optionally repeated one or more times (e.g., the variant(s) undergo a session of random mutation, the variants generated from this session of mutation are analyzed via YSD, and the variant(s) with the best binding and/or binding selectivity to the target of interest are selected). As many repetitions can be done as desired and/or as required to achieve the desired binding constant and/or binding selectivity.

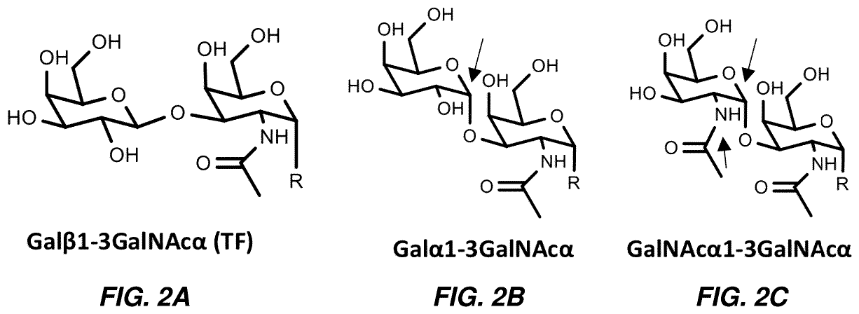

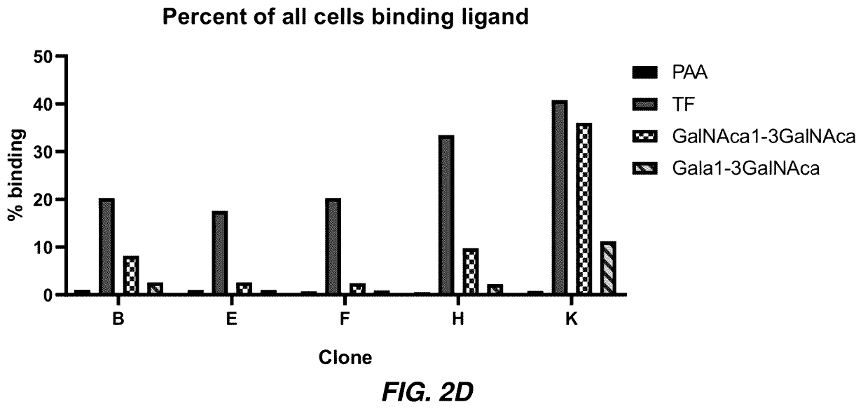

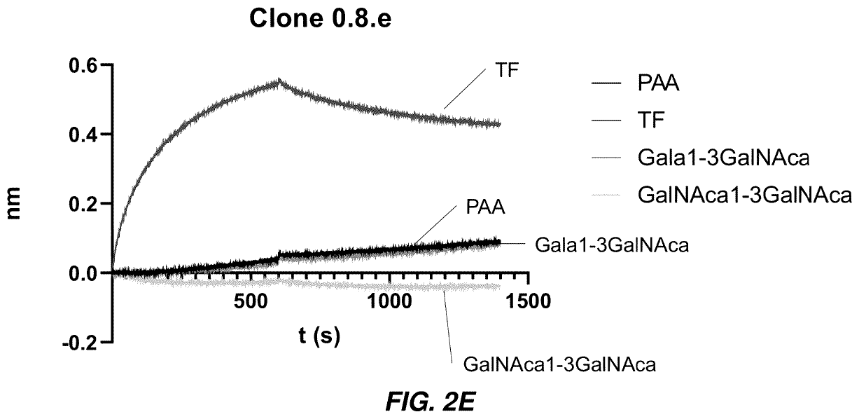

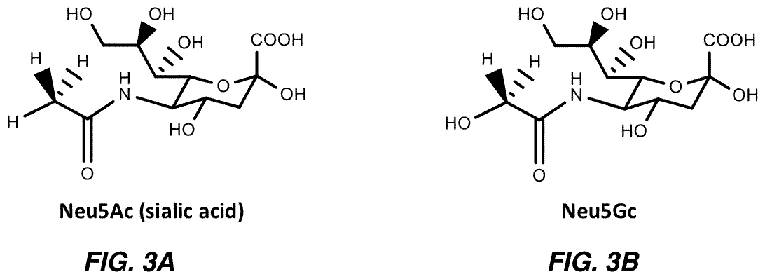

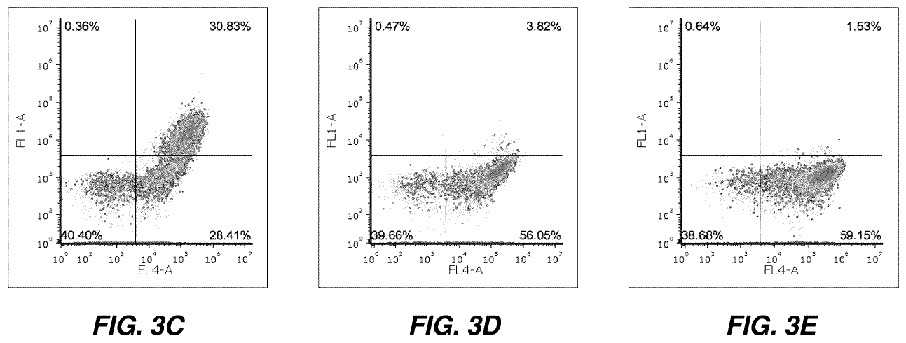

Based on techniques such as these, or others described herein, modified Sso7d proteins can be developed that can bind to various glycans, for example, but not limited to, a disaccharide (e.g. the dihexose Galβ1-3GalNAcα, also named the TF antigen, FIG. 2A) or a monosaccharide (e.g. the nonulosonic acid named Neu5Ac, FIG. 3A) and certain embodiments of the invention are also generally directed to such modified Sso7d proteins. In some cases, the binding may be relatively specific, for example, with a K_{D} of less than 10⁻⁵ M, or other values such as those described herein.

In certain embodiments, glycan-binding proteins such as those discussed herein can be used in various applications. In some cases, the protein can be modified further. For example, a glycan-binding protein could be attached to another glycan-binding protein to, for example, increase the binding and/or binding selectivity even further. As another example, in certain instances, a glycan-binding protein could be attached to another structure (e.g., a fluorophore) to, for example, functionalize the protein for a particular use, such as use for ELISAs, therapeutics, glycan characterization, cell selection, flow cytometry, histology, imaging, arrays, affinity purification, and/or enzyme-linked visualization, among other applications. A variety of applications involving the binding of a glycan to a glycan-binding protein, e.g., specifically, thus may be realized.

The above discussion illustrates various non-limiting examples of some embodiments. However, other embodiments of glycan-binding proteins and compositions thereof are also possible, as discussed below.

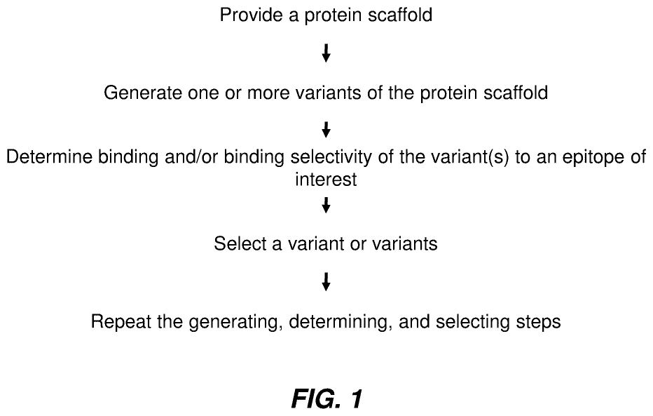

Certain aspects are related to systems and methods for producing glycan-binding proteins and compositions thereof. Non-limiting examples of such glycan-binding proteins are discussed below. Exemplary directed evolution methods of producing glycan-binding proteins are described in relation to FIG. 1. However, it should be understood that the methods described herein have broader utility, and are not limited to generating the glycan-binding proteins described herein. In addition, it should be understood that other methods may be used instead of the methods described in FIG. 1, including other directed evolution methods as well as other methods, such as ab initio calculations, to produce glycan-binding proteins and other proteins such as those described herein.

Thus, some embodiments are generally directed to directed evolution method of producing a protein, such as a glycan-binding protein. As an example of a directed evolution method, in FIG. 1, the method comprises providing a protein scaffold and generating one or more variants of the scaffold, determining binding and/or selectivity of those variants (for example, to a binding determinant of interest, such as to a monosaccharide and/or disaccharide) and selecting those that meet desired criteria (e.g., improved binding and/or selectivity). These steps can be repeated in some cases.

Certain methods, including certain directed evolution methods, start with the identification of a suitable protein scaffold. The protein scaffold may then be randomly mutated under directed evolution to produce a protein having one or more desired characteristics, such as the ability to bind a glycan, in some cases specifically.

In some cases, the protein scaffold may be one that has a binding site (e.g., a binding pocket) that has dimensions compatible with monosaccharide and/or disaccharide binding, and/or have a structure that has dimensions similar to those of any monosaccharide or disaccharide motif of interest within a glycan

In some cases the binding site may be one that is evolvable, e.g., as the protein scaffold is evolved using directed evolution. For example, the protein scaffold may be one that has a binding site (e.g., a binding pocket) that has dimensions compatible with monosaccharide and/or disaccharide binding, and/or have a binding site (e.g., a binding pocket) that has dimensions similar to those of any monosaccharide or disaccharide motif of interest within a glycan

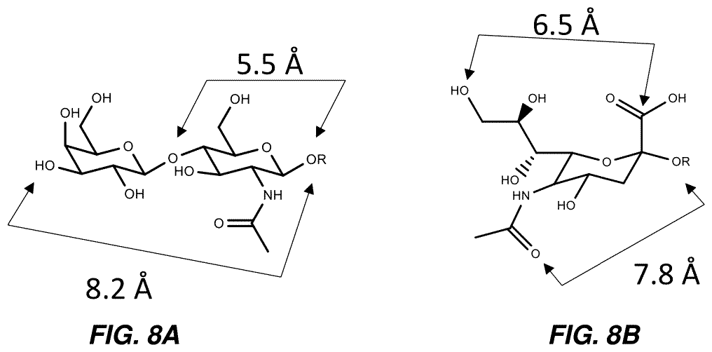

Examples of such dimensions are shown in FIGS. 8A-8B; in FIG. 8A, the dimensions of a typical disaccharide (the dihexose Galβ1-3GalNAcα) are shown; in FIG. 8B, the dimensions of a typical monosaccharide (the nonulosonic acid Neu5Ac) are shown. It should be understood that these dimensions are exemplary, and that other monosaccharides or disaccharides will have dimensions slightly different from these. However, the dimensions of the binding site of the protein scaffold may have dimensions comparable to these. For example, the binding site may have a largest dimension that is smaller than 30 Angstroms, smaller than 25 Angstroms, smaller than 20 Angstroms, smaller than 15 Angstroms, smaller than 10 Angstroms, smaller than 9.8 Angstroms, smaller than 9.6 Angstroms, smaller than 9.4 Angstroms, smaller than 9.2 Angstroms, smaller than 9.0 Angstroms, smaller than 8.8 Angstroms, smaller than 8.6 Angstroms, smaller than 8.4 Angstroms, smaller than 8.2 Angstroms, smaller than 8.0 Angstroms, smaller than 7.8 Angstroms, smaller than 7.6 Angstroms, smaller than 7.4 Angstroms, smaller than 7.2 Angstroms, smaller than 7.0 Angstroms, etc.

In some cases, the protein scaffold may be selected to have a binding face area of at least 1, at least 2, at least 3, at least 4, at least 5, at least 6, or at least 7 square nanometers (nm²). The protein scaffold, in some instances, has a binding face area of less than or equal to 6, less than or equal to 5, less than or equal to 4, or less than or equal to 3 square nanometers (nm²). Combinations of these ranges are also possible (e.g., 2-6 square nanometers (nm²)). The binding face area can be calculated by looking at the binding site of the protein scaffold, finding the longest dimension of that site, and multiplying it by the dimension of the site at a 90 degree angle from the longest dimension. For example, if the longest dimension is 30 Angstroms and the orthogonal dimension is 15 Angstroms, then the binding face area would be 450 Angstroms² (1.5×3.0) or 4.5 nm².

The protein scaffold itself may, in some cases, be one that is based on a relatively small protein, for example, one that is slightly greater than these dimensions. This may, for example, allow for multiple scaffolds to be conjugated together with minimal additional sequences. For example, the protein scaffold may be one that has a relatively low number of amino acids, e.g., less than 250 amino acids. In certain cases, the protein scaffold has less than or equal to 200 amino acid residues, less than or equal to 175 amino acid residues, less than or equal to 150 amino acid residues, less than or equal to 125 amino acid residues, less than or equal to 100 amino acid residues, or less than or equal to 75 amino acid residues. In accordance with some embodiments, the protein scaffold has greater than or equal to 25 amino acid residues, greater than or equal to 50 amino acid residues, greater than or equal to 75 amino acid residues, greater than or equal to 100 amino acid residues, or greater than or equal to 150 amino acid residues. Combinations of these ranges are also possible (e.g., the protein scaffold may have between 50-100 amino acid residues, between 50-75 amino acid residues, between 75-100 amino acid residues, or the like).

In certain instances, the protein scaffold has a maximum dimension of less than or equal to 200 Angstroms, less than or equal to 150 Angstroms, less than or equal to 100 Angstroms, less than or equal to 50 Angstroms, less than or equal to 40 Angstroms, less than or equal to 30 Angstroms, less than or equal to 25 Angstroms, less than or equal to 20 Angstroms, less than or equal to 15 Angstroms, less than or equal to 10 Angstroms, less than or equal to 7 Angstroms, or less than or equal to 3 Angstroms. In addition, according to some embodiments, the protein scaffold has a maximum dimension of greater than or equal to 5 Angstroms, greater than or equal to 9 Angstroms, greater than or equal to 12 Angstroms, greater than or equal to 15 Angstroms, greater than or equal to 18 Angstroms, greater than or equal to 20 Angstroms, greater than or equal to 25 Angstroms, greater than or equal to 30 Angstroms, greater than or equal to 40 Angstroms, etc. Combinations of these ranges are also possible (e.g., the protein scaffold may have a maximum dimension of between 15-20 Angstroms, between 20-25 Angstroms, between 10-30 Angstroms, etc.).

In addition, in some embodiments, the protein scaffold may be substantially devoid of disulfides or cysteine residues. Cysteines may cause problems with respect to disulfide bond formation, which can significantly alter the molecular structure of the protein scaffold, e.g., during the directed evolution process. For example, there may be no more than 4, 3, 2, or 1 cysteines within the protein scaffold. In some cases, no cysteines are present. Similarly, the protein scaffold may have fewer than or equal to 2, or 1 disulfide bonds, or the protein scaffold may be free of disulfide bonds.

In some cases, the protein scaffold may be selected to have a relatively high melting temperature (Tₘ), i.e., the protein scaffold may exhibit high thermal stability. For example, the protein scaffold may exhibit a melting temperature of greater than or equal to 50° C., greater than or equal to 60° C., greater than or equal to 70° C., greater than or equal to 80° C., greater than or equal to 90° C. greater than or equal to 100° C., greater than or equal to 125° C., greater than or equal to 150° C., etc. In some cases, the melting temperature may be less than or equal to 150° C., less than or equal to 125° C., less than or equal to 100° C., less than or equal to 90° C., or less than or equal to 80° C. Combinations of these ranges are also possible (e.g., 60° C. to 125° C. (inclusive)). The melting temperature or melting point is generally the temperature at which the protein begins to denature or lose its shape or 3D conformation. Accordingly, melting temperature can be determined, for example, by increasing the temperature and observing any changes in three-dimensional structure using circular dichroism (CD), differential scanning calorimetry (DSC) measurements, or the like.

The protein scaffolds may also be selected to be stable to a wide range of pH conditions. For example, the protein scaffold may be stable at a pH of greater than or equal to 1, greater than or equal to 2, greater than or equal to 3, greater than or equal to 4, greater than or equal to 5, or greater than or equal to 6. In some embodiments, the protein scaffold may be stable at a pH of less than or equal to 12, less than or equal to 11, less than or equal to 10, less than or equal to 9, or less than or equal to 8. Combinations of these ranges are also possible. For example, in some cases, the protein and/or the protein scaffold used to generate a glycan-binding protein are stable within a pH of between 2-11, or within a pH between 1-12. pH stability can be determined, for example, by adjusting the pH of the solution and observing changes in three-dimensional structure (e.g., using CD) after 30 minutes.

In some cases, a protein scaffold may be selected to be readily functionalized chemically or conjugated to other entities, for example, to generate clustered or branched assemblies. For example, the protein scaffold may be one that is capable of chemical functionalization, array display, and/or conjugation. This may be useful, for example, to generate clustered and branched assemblies to exploit avidity effects, which can be important in glycan binding in some cases. In certain embodiments, the size of the protein scaffold may be sufficiently compact, e.g., having the dimensions as discussed above, so that non-binding components of the scaffold do not substantially interfere with conjugation of glycan readers for binding multivalent glycans and more complex glycan targets. For example, in some embodiments, two protein scaffolds may be linked or conjugated together, e.g., to bind to more complex glycan targets. In some cases, the protein scaffold may be selected to be amenable to high-yield protein expression in Escherichia coli and facile bioconjugation to fluorophores, purification tags, biocompatible resins, 2-dimensional (2D) arrays, or the like. In addition, in some embodiments, the protein scaffold may be selected to be compatible with yeast surface display, in the presence and/or in the absence of Ca²⁺ or any other metal ion or cofactor.

Examples of protein scaffolds that may be suitable to produce glycan-binding proteins, such as those discussed herein, include Affibody, Fn3 domain, DARPins, Lambody, and Sso7d, these are summarized in Table 1.

TABLE 1

| SCAFFOLD | # Residues | WT Tₘ (° C.) |
|---|---|---|
| Affibody | 58 | 78 |
| Fn3 domain | 94 | 84 |
| DARPins | 130-190 | variable |
| Lambody | 217 | n/d |
| Sso7d | 63 | 98 |

Thus, in one set of embodiments, the protein scaffold may be Sso7d (e.g., from Sulfolobus solfataricus), or variants thereof. Sso7d has the following sequence:

(SEQ ID NO: 2)
ATVKFKYKGEEKQVDISKIKKVWRVGKMISFTYDEGGGKTGRGAVSEK
DAPKELLQMLEKQK

In addition, the protein scaffold may be based on the reduced-charge variant of Sso7d (rcSso7d), for example, comprising the following sequence:

(SEQ ID NO: 1)
ATVKFTYQGEEKQVDISKIKKVWRVGQMISFTYDEGGGATGRGAVSEKD
APKELLQMLEKQ.

Thus, in certain cases, the protein scaffold may be based on Sso7d or rcSso7d, with 10, 9, 8, 7, 6, 5, 4, 3, 2, or 1 changed residues. In some cases, the protein scaffold may be based on rcSso7d, but with greater than or equal to 70%, greater than or equal to 80%, greater than or equal to 90%, greater than or equal to 95%, or greater than or equal to 99% homology. The protein scaffold may also have less than or equal to 99%, less than or equal to 95%, less than or equal to 90%, or less than or equal to 85% homology to Sso7d or rcSso7d. Combinations of these ranges are also possible (e.g., 90-99% homology).

In certain embodiments, the method comprises generating one or more variants of the protein scaffold, e.g., as is shown in FIG. 1. Any number of variants may be generated. In addition, a variety of methods may be used to generate variants of the protein scaffold. For example, in some embodiments, error-prone PCR can be used to mutate the protein scaffold randomly. Other non-limiting examples include various experimental techniques (such as error-prone PCR, chemical mutagenesis, UV irradiation, etc.), or computer-based approaches (e.g., altering the amino acid sequence, e.g., randomly or with particular mutations, such as relatively conservative mutations). In some cases, site-directed mutagenesis techniques may be used (e.g., focused on one or more of the variable residue portions of a protein scaffold, such as those discussed herein). In other cases, the mutations may be randomly generated, e.g., without regard to any particular focus within the protein scaffold.

In some embodiments, the variants of the protein scaffold that are generated include, on average, greater than or equal to 1 amino acid, greater than or equal to 2 amino acids, greater than or equal to 3 amino acids, greater than or equal to 5 amino acids, etc., in each round of mutation. In certain embodiments, there may be less than or equal to 5 amino acids, less than or equal to 4 amino acids, less than or equal to 3 amino acids, or less than or equal to 2 amino acids that were mutated in a protein scaffold in a round of mutation. Combination of these ranges are also possible. In some cases, the number of mutations in a protein scaffold may not be deterministic, i.e., in techniques, such as error-prone PCR, that generate random mutations within a protein scaffold.

In some cases, the variant protein scaffolds may be studied to determine which ones exhibit desired characteristics. For example, the variants exhibiting increased binding and/or binding selectivity to the target of interest (e.g., the monosaccharide or disaccharide-binding determinant) may be determined. In some embodiments, binding and/or binding selectivity of the one or more variants to a target of interest, such as a glycan, may be used. Examples of potential targets include monosaccharide or disaccharide-binding determinants, more complex carbohydrates, or the like, e.g., as discussed herein.

For example, in accordance with certain embodiments, binding and/or binding selectivity may be determined based on binding of the variants to a target of interest, such as a monosaccharide or disaccharide-binding determinant. Non-limiting examples of monosaccharide-binding determinants include hexoses (e.g., glucose, galactose, fructose, etc.), hexosamines (e.g. glucosamine, galactosamine), heptoses or heptuloses (e.g., sedoheptulose, mannoheptulose, L-glycero-D-manno-heptose, etc.), octoses or octulosonic acids (e.g., methylthiolincosamide), nonoses or nonulosonic (sialic) acids (e.g., Kdn, Neu5Gc, Neu, Neu2en5Ac), and Neu5Ac (sialic acid) etc., as well as derivatives thereof having one or more additional substitutions at the hydroxyl groups, e.g., on C-4, C-7, C-8, and/or C-9 (such as O-acetyl, O-methyl, 0-sulfate, O-lactyl, or phosphate groups, etc.), octulosonic acids and derivatives thereof (e.g. KDO or keto-deoxyoctulosonate), and nonulosonic acids and derivatives thereof (e.g. Leg or legionaminic acid, Pse or pseudaminic acid, etc.). Non-limiting examples of disaccharide-binding determinants include dihexoses (e.g., sucrose, lactose, maltose, etc.), diheptoses, and Galβ1-3GalNAcα (TF or Thomsen-Friedenrich antigen). Those of ordinary skill in the art will be familiar with other monosaccharide or disaccharide-binding determinants as well that can be used in other embodiments, e.g., as a target of interest. Many of these have been widely discussed in the scientific literature.

Thus, one or more variants may be selected that exhibit increased binding and/or binding selectivity to a target, such as a monosaccharide or disaccharide-binding determinant. In some cases, for example, variants exhibiting improved binding (e.g., as measured by the dissociation constant or K_{D}) may be selected, for example, improvements of at least 5% or at least 10% in K_{D} in a given round of mutation/selection. It will be understood that generally, higher affinities produce smaller K_{D} values, as discussed below. Thus, such improved variants can be determined by determining K_{D} values, and selecting those that meet some suitable criteria, e.g., by selecting variants that have less than a certain K_{D} value, by selecting a certain number or percentage of variants as ranked by their K_{D} values, or the like (e.g., the 5% or 10% of variants with the lowest K_{D} values, etc.).

In some cases, variants that are selected may be those that are able to specifically bind to a target, such as a glycan. For example, specific binding may be observed with K_{D} values of less than 10⁻⁵ M, less than 10⁻⁶ M, less than 10⁻⁷ M, less than 10⁻⁸ M, less than 10⁻⁹ M, less than 10⁻¹⁰ M, etc.

A variety of methods of determining K_{D} values can be used, e.g., based on the glycan or other target. For example, one suitable technique is yeast-surface display (YSD), e.g., using with magnetic bead-immobilized glycans as discussed below. The yeast (and the variants) can be sorted, for example, using fluorescence-activated cell sorting (FACS) or other flow cytometry techniques. Other non-limiting examples include expression in alternative systems (e.g. bacteria, insect cells, mammalian cells, or the like), biolayer interferometry traces, surface plasmon resonance (SPR) traces, binding to immobilized glycan arrays, or the like. In addition, it should be understood that other methods of determining binding or selectively may be used, instead of and/or in in addition to determining K_{D} values.

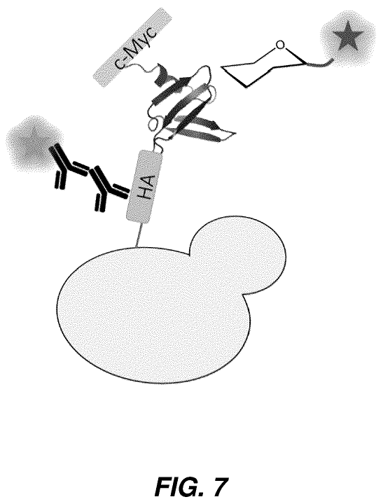

Thus, in some embodiments, the determination and/or selection are accomplished using Yeast-Surface Display (YSD) selections with magnetic bead-immobilized glycans. For example, in FIG. 7, yeast-surface display is used to determine whether a variant binds a sugar-binding determinant of interest (e.g., a monosaccharide or disaccharide-binding determinant). Moreover, in certain embodiments, YSD will be used in the presence or in the absence of Ca²⁺ or other metal ion or cofactor. Accordingly, in some cases, the protein scaffold is compatible with YSD in the presence of Ca²⁺ and/or in the absence of Ca²⁺.

In certain embodiments, the above steps (e.g., generating, determining, and selecting) may be repeated, using the variant exhibiting increased binding and/or binding selectivity as the next protein scaffold that binds to the target (e.g., a monosaccharide or disaccharide-binding determinant) in each repeat. In some instances, the generating, determining, and selecting steps are repeated, for example, until one or more variants with the desired binding and/or binding selectivity is obtained, e.g., as discussed herein. In some embodiments, these steps are repeated at least once, at least 5 times, at least 10 times, at least 20 times, or more in some cases. In certain instances, these steps are repeated less than or equal to 25 times, less than or equal to 20 times, less than or equal to 10 times, less than or equal to 5 times, or less than or equal to 2 times. Combinations of these ranges are also possible (e.g., 1-2 times).

In certain cases, once the variant has been characterized and/or its sequence has been identified, the generated protein can then be made with other common techniques available in the art. For example, the protein could be synthesized or it could be expressed in cells, such as in E. coli. Those of ordinary skill in the art will be aware of systems and methods for expressing a protein from its nucleic acid sequence.

Another aspect of the present invention is generally related to glycan-binding proteins and compositions thereof, e.g., generated using the techniques discussed above, or other techniques. The protein, in accordance with certain embodiments, may be able to bind to a glycan-binding determinant including any of those described herein e.g., via specific binding. For example, the protein may exhibit binding to a monosaccharide or a disaccharide-binding determinant, e.g., with K_{D} values of less than 10⁻⁵ M, less than 10⁻⁶ M, less than 10⁻⁷ M, less than 10⁻⁸ M, less than 10⁻⁹ M, less than 10⁻¹⁰ M, etc. In addition, the protein can exhibit selective binding to a target glycan in certain embodiments, e.g. as compared to other glycans having similar structures. For example, the protein may be able to tightly bind to single copies of a binding determinant and/or distinguish differences at the atomic level.

As an example, as discussed, certain glycan-binding proteins are generally based on rcSso7d used as a protein scaffold. Native Sso7d is a DNA-binding protein, but does not significantly bind glycans. It forms an SH3-domain-like fold with five beta (β)-strands and an alpha (α)-helix at the C-terminus. In certain embodiments, the protein rcSso7d has a similar, or identical, three-dimensional structure to that of native Sso7d. For example, in certain cases, the protein has an SH3-domain-like fold. The protein, in some instances, has five beta (β)-strands. The protein has an alpha (α)-helix at the C-terminus, in certain embodiments. The three-dimensional structure of the protein may be considered similar to that of Sso7d if it has one or more of (i) an SH3-domain-like fold, (ii) five beta (β)-strands, or (iii) an alpha (α)-helix at the C-terminus.

In some cases, for example, the glycan-binding protein may exhibit a certain degree of homology to Sso7d (SEQ ID NO: 2), or to modified Sso7d sequences such as those described herein, for instance, the reduced-charge variant of Sso7d (rcSso7d) shown as SEQ ID NO: 1. For instance, the glycan-binding protein may exhibit 50% or greater, 55% or greater, 60% or greater, 65% or greater, 68% or greater, 70% or greater, 75% or greater, 80% or greater, 85% or greater, 90% or greater, 95% or greater homology, 97% or greater, or 99% to one or more of the sequences disclosed herein, for example, Sso7d, a modified Sso7d such as the reduced-charge variant of Sso7d (rcSso7d) of SEQ ID NO: 1, or other scaffold protein such as affibodies, Fn3 domains, DARPins, Lambodies, or the like. The glycan-binding protein may also have 99% or less, 95% or less, 90% or less, 85% or less, 80% or less, 75% or less, 70% or less, 65% or less, or 60% or less homology to one or more of those sequences. Combinations of these ranges are also possible (e.g., 55-90% homology, 68-90% homology, 75-90% homology, and 75-85% homology, etc.). As mentioned, there may be variants from the original scaffold protein, e.g., caused by directed evolution or other techniques descried herein, that allow the protein to bind to glycans. Thus, in some cases, the homology may exclude 100% (i.e., exclude wild-type scaffold proteins), since such proteins may not be able to bind glycans, or bind to glycans very poorly.

In some embodiments, the glycan-binding protein may have at least 1, at least 2, at least 3, at least 4, at least 5, at least 6, at least 8, at least 10, at least 12, at least 14, at least 16, at least 18, at least 20, at least 22, at least 24, at least 26, at least 28, at least 30, at least 32, at least 34, at least 36, at least 38, and/or no more than 40, no more than 38, no more than 36, no more than 34, no more than 32, no more than 30, no more than 28, no more than 26, no more than 24, no more than 22, no more than 20, no more than 18, no more than 16, no more than 14, no more than 12, no more than 10, no more than 8, no more than 6, no more than 5, no more than 4, no more than 3, or no more than 2 mutations relative to the initial scaffold protein, e.g., to Sso7d, a modified Sso7d such as the reduced charge variant of Sso7d (rcSso7d) of SEQ ID NO: 1, or other scaffold protein such as affibodies, Fn3 domains, DARPins, Lambodies, or the like. As a non-limiting example, a scaffold protein may have 2-4, 6-8, or 10-14 mutations relative to SEQ ID NO: 1 or SEQ ID NO: 2.

In addition, in some cases, the glycan-binding protein may have at least 34 amino acids, at least 37 amino acids, at least 40 amino acids, at least 43 amino acids, at least 46 amino acids, at least 49 amino acids, at least 52 amino acids, at least 55 amino acids, or at least 58 amino acids of one or more of the sequences in the same order. In certain embodiments, the protein may have 61 or fewer amino acids, 58 or fewer amino acids, 55 or fewer amino acids, 52 or fewer amino acids, 49 or fewer amino acids, 46 or fewer amino acids, 43 or fewer amino acids, 40 or fewer amino acids, or 37 or fewer amino acids of one or more of the sequences disclosed above in the same order. Combinations of these ranges are also possible (e.g., 37-58 amino acids of the sequences disclosed above in the same order).

In some embodiments, the amino acids may be contiguous or noncontiguous. For example, the following sequence (discussed in Example 2, Sequence List 1) has 45 amino acids (shown in underlining) of SEQ ID NO: 1:

(SEQ ID NO: 14)
ATVKFTYRGEEKQVGVSRVKSVHRIGQWIKFWYDEGSGAYGRGYVSEK
DAPEELLQMLEKRGSEQKLISEEDL.

Notably, in this example, some of the homologous amino acids are contiguous (e.g., the following 7 amino acid stretch: ATVKFTY (SEQ ID NO: 15)) while others are noncontiguous (e.g., the following 8 homologous amino acids in an 18 amino acid stretch: GVSRVKSVHRIQQWIKFW (SEQ ID NO: 16)).

In some cases, there may be additional amino acids that are not present in the protein scaffold, before, after, and/or in between contiguous sections. For example, in the above example, the protein has 12 amino acids at the end of its sequence that are not present in the protein scaffold (SEQ ID NO: 1). Similarly, in certain instances, there may be sections of the protein scaffold that are missing from the protein. For example, in the above example, the protein contains the sequence OGVSRVKSV (SEQ ID NO: 410) while the protein scaffold (SEQ ID NO: 1) contains the sequence OVDISKIKKV (SEQ ID NO: 411). In this case, the protein scaffold has an extra amino acid (11 amino acids compared to 10 amino acids). Lastly, in this example, since there are 45 amino acids of the protein scaffold in the protein, 62 amino acids in the protein scaffold, and 73 amino acids in the protein, the protein has 72.6% (45/62) homology to the protein scaffold (SEQ ID NO:1).

As mentioned, certain embodiments of the invention are generally directed to modified Sso7d sequences that are able to bind to a glycan, for instance specifically. In some instances, the protein may be able to bind to a monosaccharide or disaccharide-binding determinant. For example, in some cases, the Sso7d, or a reduced charge variant thereof, may be modified in one or more surface-exposed residues on the protein. For instance, in one set of embodiments, 1, 2, 3, 4, 5, 6, 7, 8, or 9 or more surface-exposed residues may be modified. As a specific non-limiting example, certain embodiments of the invention are generally directed to the following sequence:

ATVKFTYQGEEKQVDISKIKKX¹VX²RX³GQX⁴IX⁵FX⁶YDEGGGAX⁷GX⁸G
X⁹VSEKDAPKELLQMLEKQ,

where each of X¹, X², X³, X⁴, X⁵, X⁶, X⁷, X⁸, and X⁹ is independently an amino acid residue, with the proviso that X¹, X², X³, X⁴, X⁵, X⁶, X⁷, X⁸, and X⁹ cannot all be K, W, V, M, S, T, T, R, and A, respectively (SEQ ID NO: 4). However, it should be understood that individually, one or more of these substitutions may still be made, e.g., 1, 2, 3, 4, 5, 6, 7, or 8 of the substitutions of X¹ with K, X² with W, X³ with V, X⁴ with M, X⁵ with S, X⁶ with T, X⁷ with T, X⁸ with R, and X⁹ with A can be made in various embodiments.

In addition, other embodiments of the invention are generally directed to sequences that are homologous to any of the above sequences, e.g., sequences exhibiting 50% or greater, 55% or greater, 60% or greater, 65% or greater, 68% or greater, 70% or greater, 75% or greater, 80% or greater, 85% or greater, 90% or greater, 95% or greater homology, 97% or greater, and/or 99% or less, 95% or less, 90% or less, 85% or less, 80% or less, 75% or less, 70% or less, 65% or less, or 60% or less homology to this sequence. Combinations of these ranges are also possible (e.g., 55-90% homology, 68-90% homology, 75-90% homology, and 75-85% homology, etc.).

In certain cases, the protein may be a modified Sso7d sequences that are able to bind to a glycan, e.g. specifically. For example, the protein may be able to bind to a monosaccharide or disaccharide-binding determinant. In one embodiment, the protein has the following sequence:

(SEQ ID NO: 3)
ATVKFTYQGEEKQVDISKIK(s1)(s2)DEGGG(s3)SEKDAPKELLQML
EKQ.

In this sequence, (s1), (s2), and (s3) represent regions of a reduced charge Sso7d variant that are surface-exposed, and may be modified. For example, independently within each of (s1), (s2), and (s3), 1, 2, 3, 4, 5, 6, or 7 of the amino acid residues within these sequences may be modified. In the initial variant, (s1) is KKVWRVG (SEQ ID NO: 407), (s2) is QMISFTY (SEQ ID NO: 408), and (s3) is ATGRGAV (SEQ ID NO: 409), and one or more of (s1), (s2), and (s3) may be modified, e.g., to have a sequence different than these. Thus, for example, in one embodiment, (s1) consists of 7 amino acid residues and is not KKVWRVG (SEQ ID NO: 407), (s2) consists of 7 amino acid residues and is not QMISFTY (SEQ ID NO: 408), and (s3) consists of 7 amino acid residues and is not ATGRGAV (SEQ ID NO: 409).

In some embodiments, 1, 2, or 3 of positions 2, 4, and 6 of (s1) may be modified, e.g., with a different amino acid residue, for example, as in KX¹VX²RX³G (SEQ ID NO: 412), where each of X¹, X², and X³ independently are amino acid residues, although X¹, X², and X³ cannot simultaneously be K, W, and V, respectively. In some embodiments, 1, 2, or 3 of positions 2, 4, and 6 of (s2), e.g., with a different amino acid residue, for example, as in QX⁴IX⁵FX⁶Y (SEQ ID NO: 413), where each of X⁴, X⁵, and X⁶ independently are amino acid residues, although X⁴, X⁵, and X⁶ cannot simultaneously be M, S, and T. In some embodiments, 1, 2, or 3 of positions 2, 4, and 6 of (s3), e.g., with a different amino acid residue. In addition, in certain cases, the substitution is not with cysteine, for example, as in AX⁷GX⁸GX⁹V (SEQ ID NO: 414), where each of X⁷, X⁸, and X⁹ independently are amino acid residues, although X⁷, X⁸, and X⁹ cannot simultaneously be T, R, and A.

In addition, other embodiments of the invention are generally directed to sequences that are homologous to any of the above-described sequences, e.g., sequences exhibiting 50% or greater, 55% or greater, 60% or greater, 65% or greater, 68% or greater, 70% or greater, 75% or greater, 80% or greater, 85% or greater, 90% or greater, 95% or greater homology, 97% or greater, and/or 99% or less, 95% or less, 90% or less, 85% or less, 80% or less, 75% or less, 70% or less, 65% or less, or 60% or less homology to this sequence. Combinations of these ranges are also possible (e.g., 55-90% homology, 68-90% homology, 75-90% homology, and 75-85% homology, etc.).

Non-limiting examples of such proteins include those described in Sequence List 1 and Sequence List 2 (shown in Example 2).

Any of the amino acid substitutions described anywhere herein may be a substitution with natural and/or unnatural amino acids, and may include 1 or 2, 3, 4, etc., amino acids that are substituted in. Those of ordinary skill in the art will be aware of amino acids. For instance, the naturally-occurring amino acids include are the 20 amino acids most commonly found in nature, typically in the L-isomer, i.e., alanine (“Ala” or “A”), arginine (“Arg” or “R”), asparagine (“Asn” or “N”), aspartic acid (“Asp” or “D”), cysteine (“Cys” or “C”), glutamine (“Gln” or “Q”), glutamic acid (“Glu” or “E”), glycine (“Gly” or “G”), histidine (“His” or “H”), isoleucine (“Ile” or “I”), leucine (“Leu” or “L”), lysine (“Lys” or “K”), methionine (“Met” or “M”), phenylalanine (“Phe” or “F”), proline (“Pro” or “P”), serine (“Ser” or “S”), threonine (“Thr” or “T”), tryptophan (“Trp” or “W”), tyrosine (“Tyr” or “Y”), and valine (“Val” or “V”). In some embodiments, only natural amino acids are used in the protein.

However, in some cases, one or more unnatural amino acids may be present. An unnatural amino acid is an amino acid (or an imino acid) that is not one of the 20 natural amino acids. Non-limiting examples of unnatural amino acids include D-isomers of the natural amino acids (with the exception of glycine, which is identical to its L-isomer), as well as other amino acids such as alloisoleucine, allothreonine, homophenylalanine, homoserine, homocysteine, 5-hydroxylysine, 4-hydroxyproline, 4-carboxyglutamic acid, cysteic acid, cyclohexylalanine, ethylglycine, norleucine, norvaline, 3-aminobutyric acid, beta-amino acids (e.g., beta-alanine), N-methylated amino acids such as N-methylglycine, N-methylalanine, N-methylvaline, N-methylleucine, N-methylisoleucine, N-methylnorleucine, N-methyl-2-aminobutyric acid, N-methyl-2-aminopentanoic acid, etc.

In some cases, the glycan-binding protein may have a relatively high melting temperature (Tₘ) or exhibit high thermal stability. For example, the glycan-binding protein may exhibit a melting temperature of greater than or equal to 50° C., greater than or equal to 60° C., greater than or equal to 70° C., greater than or equal to 80° C., greater than or equal to 90° C. greater than or equal to 100° C., greater than or equal to 125° C., greater than or equal to 150° C., etc. In some cases, the melting temperature may be less than or equal to 150° C., less than or equal to 125° C., less than or equal to 100° C., less than or equal to 90° C., or less than or equal to 80° C. Combinations of these ranges are also possible (e.g., 60° C. to 125° C. (inclusive)).

The glycan-binding protein may also be stable to a wide range of pH conditions. For example, the glycan-binding protein may be stable at a pH of greater than or equal to 1, greater than or equal to 2, greater than or equal to 3, greater than or equal to 4, greater than or equal to 5, or greater than or equal to 6. In some embodiments, the glycan-binding protein may be stable at a pH of less than or equal to 12, less than or equal to 11, less than or equal to 10, less than or equal to 9, or less than or equal to 8. Combinations of these ranges are also possible, for example, stable within a pH of between 2-11, or within a pH between 1-12, etc.

In one embodiment, the protein is not any one of the following sequences:

(SEQ ID NO: 5)
ATVKFTYQGEEKQVDISKIKWVIRWGQHIAFKYDEGGGAAGYGWVSEK
DAPKELLQMLEKQ,
(SEQ ID NO: 6)
ATVKFTYQGEEKQVDISKIKWVNRWGQRIYFKYDEGGGAAGYGWVSEK
DAPKELLQMLEKQ,
(SEQ ID NO: 7)
ATVKYTYRGEEKRVDISKIKWVNRWGQHLAFKYDKGGGAAGYGWVSEK
DAPKELLQMLEKR,
(SEQ ID NO: 8)
ATVKSTYRGEEKQVDISKIKWVIRWGQHLAFKYDEGGGAAGYGWVSEK
DAPKELLQMLEKQ,
(SEQ ID NO: 9)
ATVKFTYRGEEKQVDISKIKWVNRWGQHLAFKYDVGGGAAGYGWMSEK
DAPKELLQMLEKR,
(SEQ ID NO: 10)
ATVKFTYQGEEKQVDISKIKWVIRLGRTIMFKYDEGGGANGYGKVSEK
DAPKELLQMLEKQ,
(SEQ ID NO: 11)
ATVKFTYQGEEKQVDISKIKWVVRLGQVIMFKYDEGGGANGYGKVSEK
DAPKELLQMLEKQ,
(SEQ ID NO: 12)
ATVKFTYRGEEKQVDISKIKWVVRLGQVIMFKYGEGGGSNGYGRVSEK
DAPKELRQMLEKR,
or
(SEQ ID NO: 13)
ATVKFTYRGEEKQVDISKIKWVVRLGQVIMFKYDEGGGASGYGRVSEK
DAPKELLQMLEK

In accordance with some embodiments, two or more proteins are linked directly to each other, or indirectly linked, e.g., by a suitable linker. Thus, in certain embodiments, the composition comprises one or more glycan-binding portions (e.g., a first glycan-binding portion and a second glycan-binding portion). The proteins can be linked, for example, C-terminus to C-terminus, N-terminus to N-terminus, C-terminus to N-terminus, or in other suitable configurations in certain instances. In some instances, the two or more proteins are joined in a linear structure. In certain cases, the two or more proteins are joined in a branched structure. In some embodiments, the two or more proteins are immobilized proximally as part of a surface immobilized array.

In some cases, two or more linked proteins may be useful to create compositions that can bind to longer glycans. For instance, a first glycan-binding portion may recognize a first binding determinant in a glycan while a second glycan-binding portion may recognize a second binding determinant in the same glycan. In this way, longer glycans comprised of more than one saccharide may be selectively bound or even sequenced in some cases, e.g., using suitable proteins such as those discussed herein. In certain embodiments, one or more of the glycan-binding portions may include protein structures such as any of these disclosed herein, for example, those generally based on Sso7d, reduced-charge variant of Sso7d (rcSso7d), etc. In some cases, such glycans may be sequenced or their identities may be determined, e.g., as discussed herein.

For example, in some cases, one or more linked proteins may be used to identify glycan structures within glycoproteins, glycolipids, glyconucleic acids, proteoglycans, or the like. For instance, glycan structures may comprise a plurality of saccharide units (e.g., Neu5Ac, Kdn, Neu5Gc, Neu, Neu2en5Ac, mannose, glucose, GlcNAc, galactose, Xyl, fucose, Leg, Pse, etc.) joined together in various configurations (e.g. by α- or β-glycosidic linkage) or onto various structures (e.g., via N-glycosylation, O-glycosylation, etc.), and the linked protein may be able to identify two, three, or more saccharide-binding determinants within such structures.

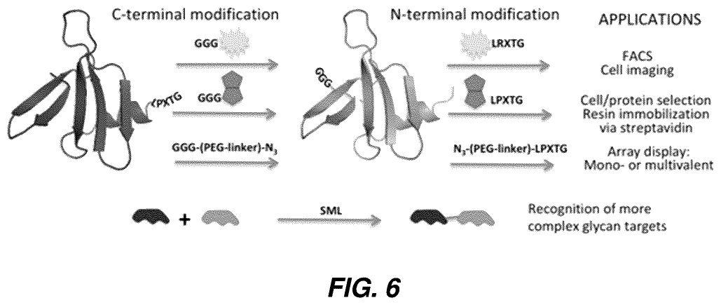

In some embodiments, the linkage between the proteins can be accomplished indirectly. The linker, in certain embodiments, comprises a peptidic linker. For example, in FIG. 6, two proteins are linked together via an LPXTG (SEQ ID NO: 17) (or LRXTG (SEQ ID NO: 18)) sequence on one of the proteins (where X can be any amino acid) and a GGG sequence on the other protein. These may be linked together, for example, using sortase or other suitable enzymes. The LPXTG (SEQ ID NO: 17) (or LRXTG (SEQ ID NO: 18)) sequence may be found near the C-terminus of a first protein and the GGG sequence may be found near the N-terminus of a second protein, and sortase may thus covalently link the N-terminus of the first protein to a location near (within ˜100 amino acids of) the C-terminus of the second protein. As another example, the peptidic linker may comprise a Gly-rich linker, e.g., a Gly-Gly linker or other Glyn linkers (n being any positive integer, e.g., 1, 2, 3, 4, 5, 6, etc.). Other amino acids may also be present in a Gly-rich linker, e.g. as in (GGGGS)ₙ (SEQ ID NOs: 19-24).

The linker, in some instances, comprises a non-peptidic linker. A variety of non-peptidic linkers can be used, including click chemistry techniques, PEG, or the like. For example, a non-peptidic linker may comprise a polyethylene glycol (PEG) linker. For example, in FIG. 6, two proteins are linked via PEG in combination with an azide-alkyne click-chemistry linker.

According to certain embodiments, two proteins may be directly linked to each other by ligating or joining their nucleic acid sequences together such that the two proteins are expressed together. For instance, the two or more proteins may be genetically fused together.

In some cases, linking two proteins together may increase binding and/or binding selectivity to the target of interest (e.g., the monosaccharide or disaccharide-binding determinant).

In accordance with some embodiments, the composition further comprises an additional structure. For example, in some cases, the additional structure comprises a protein (e.g., a non-glycan-binding protein), enzyme, affinity tag (e.g. polyHis tag) and/or an oligonucleotide sequence, and/or small molecule (for instance, having a molecular weight of less than 2000 or 1000 Da). In some embodiments, the small molecule comprises a fluorophore. For example, in FIG. 6, one of the proteins is attached to a fluorophore.

The additional structure may be covalently attached to the protein, in certain instances. For example, in some instances, the additional structure is covalently attached to the protein via multivalent dendritic polymer backbones. According to certain embodiments, the additional structure comprises an oligomerization domain of a native protein (e.g., a non-glycan-binding protein), and the oligomerization domain is fused to the protein.

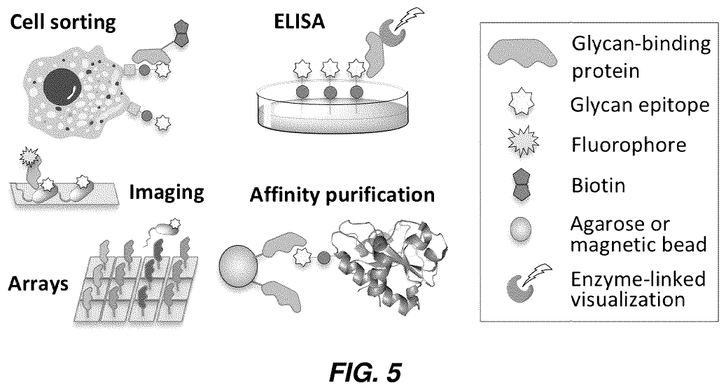

In some embodiments, the proteins, and compositions thereof, described herein have numerous applications, including in identification, manipulation, diagnostics, ELISAs, glycan characterization, cell selection, immunoblotting, flow cytometry, histology, imaging, arrays, affinity purification, and/or enzyme-linked visualization. For example, FIG. 5 shows some possible uses, in some cases, for the glycan-binding proteins, and compositions thereof, disclosed herein.

For instance, in some cases, the proteins disclosed herein may be useful as substitutes or analogs for antibodies and antibody-like biomolecules in immunological, therapeutic, diagnostic, or technological applications, such as flow cytometry, histology, and others. The generated proteins disclosed herein, in some instances, can be used to identify and/or manipulate a carbohydrate of interest regardless of size or composition.

Many carbohydrates or biomolecules play significant roles in various diseases, and systems and methods for determining glycans, e.g., using glycan-binding proteins such as those discussed herein, may be useful for identifying, characterizing, or sequencing such glycans. As another example, such proteins could be used to determine human cancer-binding determinants, bacterial glycans, or the like.

In certain embodiments, proteins such as those disclosed herein can be attached to other groups, providing a vast array of applications. For example, in some cases, proteins such as those disclosed herein can be attached to a fluorophore. This could be useful, for example, in imaging of a glycan-binding determinant of interest (or molecules containing the glycan-binding determinant of interest). As another example, in certain instances, a protein can be attached to a molecule such as biotin. This could be useful, for example, various in cell selection applications. According to yet another example, a protein disclosed herein can be attached to a bead, such as an agarose bead or a magnetic bead. This could be useful, for example, in affinity purification of glycan-binding determinants of interest (or molecules containing the glycan-binding determinant of interest).

According to certain embodiments, the proteins (and compositions thereof) described herein have various advantages. For example, in some embodiments, the methods described herein can be used to generate a protein specific for any desired target, which can be useful, for example, where there are no native binders of that target. In some cases, the proteins described herein may be more stable (e.g., to temperature or pH) than other binders of the desired target. Moreover, in some instances, the proteins described herein are small enough that they can recognize single-atom differences between molecules (e.g., sugars), which may provide higher specificity for a target of interest than other binders, and/or which may prevent or reduce steric hindrance.

Without wishing to be bound by theory, it is believed that, in certain embodiments, generating a glycan-binding protein from a protein that does not typically bind sugars (e.g., from a DNA-binding protein or a protein-binding protein) can improve selectivity for the glycan of interest, for instance, as there is no possibility of lingering native sugar-binding functionality for a different sugar. Similarly, in some embodiments, the proteins described herein have higher binding constants for the target of interest than other binders. Further, in certain cases, the proteins described herein can be easily attached to one another (e.g., through sortase-mediated ligation or genetic fusion) or to other groups (e.g., fluorophores or chemical handles) for easy functionalization.

The following examples are intended to illustrate certain embodiments of the present invention, but do not exemplify the full scope of the invention.

### Example 1

This example describes an archaeal DNA binding protein to bind and manipulate glycans, or carbohydrates and carbohydrate-containing biomolecules. As discussed herein, small DNA-binding proteins (based on Sso7d from Sulfolobus solfataricus) can be engineered using directed evolution to bind and specifically recognize targeted monosaccharides (e.g. hexose, heptulose, octulosonic and nonulosonic derivatives), disaccharides, and other more complex carbohydrates, although wild-type Sso7d is not able to bind to any glycans. As such, the engineered proteins may be able to substitute for antibody and antibody-like biomolecules in various immunological, diagnostic, and/or technological roles, such as flow cytometry, histology, and others. The proteins directly can also be used as a protein reagent capable of identifying and manipulating a carbohydrate of interest regardless of size or composition, filling a long-standing need in the glycosciences and medicine. Importantly, the proteins can also be assembled, e.g., in a “mix-and-match” fashion, to create custom reagents.

In some embodiments, the engineered proteins can tightly bind single copies of a sugar and distinguish single differences at the atomic level. The proteins may also be capable of straightforward chemical functionalization, do not require specialized training for use, and can be linked in some cases to assemble a reagent capable of specifically recognizing and manipulating complex oligosaccharide structures.

This example describes the preparation of glycan-binding proteins from an Sso7d library. The initial Sso7d library was prepared based on the methods described in Traxlmayr, M. W. et al. J. Biol. Chem. 2016, 291(43), 22496-22508. This library was prepared from a reduced charge-variant of Sso7d, a native DNA binder. Nine surface-exposed residues on one face of a reduced-charge variant of Sso7d were randomized with 18 different amino acids (all of the 20 naturally occurring amino acids, except the original amino acid itself and cysteine to avoid any sulfide groups) to generate a combinatorial library of approximately 10⁹ Sso7d variants. This was accomplished by PCR elongation and amplification of the SSo7d gene, followed by electroporation of PCR fragments and linearized vectors into yeast.

Sso7d has the following sequence:

(SEQ ID NO: 2)
ATVKFKYKGEEKQVDISKIKKVWRVGKMISFTYDEGGGKTGRGAVSEK
DAPKELLQMLEKQK

while the reduced-charge variant of Sso7d has the following sequence:

(SEQ ID NO: 1)
ATVKFTYQGEEKQVDISKIKKVWRVGQMISFTYDEGGGATGRGAVSEK
DAPKELLQMLEKQ,

where the underlining indicates the nine residues that were randomized.

After a Sso7d library was prepared as discussed above, the Sso7d library was then panned in these experiments via yeast-surface display (YSD) selections with magnetic bead-immobilized glycans for evolution of glycan binders using established techniques for yeast display. The beads were Dynabeads, which are made of polystyrene with a ferrous core. The bead-immobilized glycans used included a dihexose (e.g. Galβ1-3GalNAcα, the TF or Thomsen-Friedenrich antigen) or a nonulosonic acid (e.g. Neu5Ac.) Glycans were added by covalent chemical conjugation via a tosyl moiety or by non-covalent interactions between a biotin molecule on the glycan and a streptavidin tetramer on the bead surface.

Variants that bound glycans of interest with higher binding and/or binding selectivity were selected. In each bead selection (three or more were performed), yeast cells displaying Sso7d were selected by (i) their ability to stay bound to magnetic beads through rigorous, iterative rounds of washing, agitation, and/or presence of competitors, and/or (ii) their inability to stay bound on beads displaying undesired molecules, such as other saccharides or polymeric backbones. Once selected by bead selections and FACS sorts, Sso7d variants on yeast surfaces were required to bind polymeric sugar reagents (sugar-PAA-FITC) in solution state and any variants that did this moved forward in the process.

The selected variants were then mutated further. Mutated residues were no longer limited to the 9 surface exposed residues in order to allow for more possibilities for favorable properties to be found, by allowing mutations throughout the protein. FACS sorting allowed identification and physical selection of the tightest binding yeast cells, and these were propagated and their expression vectors removed for DNA sequencing. This DNA material was then used in any further mutagenesis by error-prone PCR or by rational site-directed mutagenesis. The process (i.e., mutating and selecting) was repeated numerous times.

After variants of proteins exhibiting desired binding and/or binding selectivities were obtained, the genes of the Sso7d variants of interest were amplified from yeast expression vectors by PCR, and the resulting PCR fragments were cloned into an E. coli expression vector bearing an affinity tag. Proteins were overexpressed in E. coli bearing the vector and the proteins were purified by affinity chromatography and characterized by SDS-Page for identity and purity.

In some cases, the variants were then conjugated to other variants (of the same or different types) and to other structures (e.g., fluorophores). For example, some expressed Sso7d variants were elongated to contain the sequence LPXTG (SEQ ID NO: 17). They were then ligated via sortase-mediated ligation to bear short peptides carrying a biotin molecule. They have also been sortagged to contain the FITC fluorophore. Sso7d variants have been attached to each other via genetic fusion, but also are attached by sortase-mediated mediated ligation.

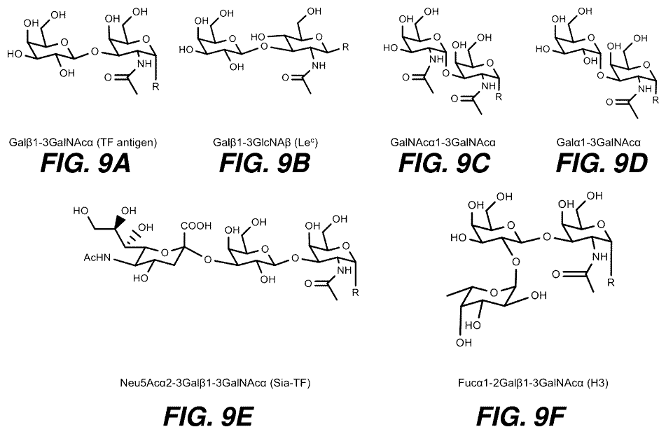

Non-limiting examples of Sso7d variants that can bind to glycans are shown below. The exemplary variants in Sequence List 1 were engineered to bind one or more nonulsonic acids, while the exemplary variants in Sequence List 2 were engineered to bind one or more dihexoses. The disaccharides (or disaccharides motifs within trisaccharides) bound by variants in Sequence List 1 and 2 are shown in FIGS. 9A-9F. Every variant listed in Sequence List 1 and Sequence List 2 bound at least one disaccharide (or disaccharide motif within a trisaccharide) in FIGS. 9A-9F. These variants are not shown in any particular order.

### Example 2

This example describes some of the glycan-binding proteins of Example 1. The protein scaffold (SEQ ID NO: 1) of Example 1 is a reduced-charge variant of Sso7d, which is a native DNA binder. The protein scaffold was used to generate the glycan-binding proteins. It had 63 residues and a melting temperature of 98° C. The protein scaffold was stable to prolonged exposure to pH values with the range of 0.3-12.5 and was free of disulfides. The protein scaffold was compatible with yeast surface display, high-yield protein expression in E. coli, and functionalization. The protein scaffold formed an SH3-domain-like fold with five beta (β)-strands and an alpha (α)-helix at the C-terminus.

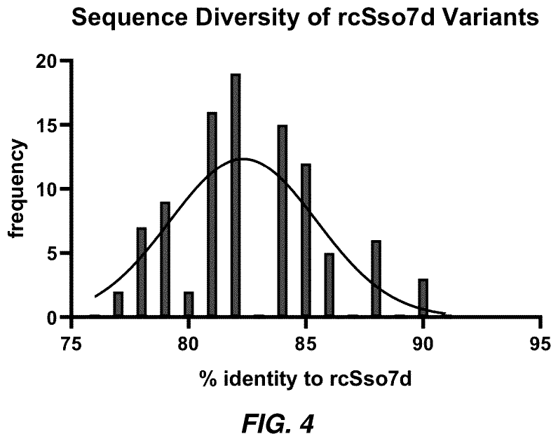

The glycan-binding proteins that were found in Example 1 were generally stable to the described biochemical manipulations and were predicted to be well-folded both on yeast surfaces and as soluble expressed proteins based the observed binding properties. Anecdotally it also is known that proteins that are efficiently expressed on yeast cell surfaces must be well-folded. In addition, the glycan-binding proteins had sequences that diverged significantly from the protein scaffold. FIG. 4 shows a histogram of the number of variants in Sequence List 2 that bind glycans versus percent homology in sequence compared to the original protein scaffold (the reduced-charge variant of Sso7d, or rcSso7d). Notably, these sequences are significantly different than the protein scaffold, with the most divergent sequences having approximately 68-69% homology. For example, these histograms include the following sequences that have 68.852% homology to the protein scaffold:

(SEQ ID NO: 13)
ATVKFTYRGEEKQVGVSRVKSVHRIGQWIKFWYDEGSGAYGRGYVSEK
DAPEELLQMLEKRGSEQKLISEEDL
(SEQ ID NO: 394)
ATVKFTYRGEEKQVGISRIRSVHRIGQWIKFWYDEGSGACGRGYVSEK
GAPKELLQMLGKRGSEQKLISEEDL
(SEQ ID NO: 395)
ATVKFTYRGKEKRVGVSRIKSVRRIGQWIKFWYDEGSGAYGRGYVSEK
DAPKELLQMLEKRGSEQKLISEEDL
(SEQ ID NO: 396)
ATVKFTYRGEEKQVGINRIKSVHRIGQWIKFWYDEGSGAYGRGYVSGK
DAPKELLRMLEKRGSEQKLISEEDL

Despite the differences in sequence, these variants are all predicted to form an SH3-domain-like fold with five beta (β)-strands and an alpha (al-helix at the C-terminus. Other glycan-binding proteins with even more divergence are also predicted to exhibit a similar SH3-domain-like fold with five beta (β)-strands and an alpha (α)-helix at the C-terminus.

### Example 3

This example describes some glycan-binding proteins from Example 1.

Some of the variants that were generated demonstrated high specificity for a target of interest and could distinguish small points of differences between molecules that were targeted and other, non-target molecules having similar structures. For instance, in this example, variants were evolved to bind Galα1-3GalNAcα (TF antigen), as discussed in Example 1. These variants demonstrated K_{D} values for the TF antigen of 3 nM to 150 nM. These variants were studied with biolayer interferometry (BLI).

FIGS. 2A-2C show the structure of the TF antigen compared to the structures of Galα1-3GalNAcα and GalNAcα1-3GalNAcα, with arrows pointing to the stereocenters and functional groups that vary from the TF antigen. Specifically, Galα1-3GalNAcα differs from the TF antigen in having a substituent in the axial position instead of an equatorial position. GalNAcα1-3GalNAcα has an additional differentiation, in that a hydroxyl group is replaced by an N-acetamide substituent.

FIG. 2D shows the percent binding of these three compounds and a sugar-polyacrylic acid (PAA)-FITC conjugate as a control for five different variants that were identified in these experiments. This binding was determined by analytical flow cytometry, wherein fluorescently labeled yeast and fluorescently labeled sugar-PAA-FITC were co-localized. These five variants (from Sequence List 2) have the following sequences:

(SEQ ID NO: 397)
ATVKFTYQGEEKQVDISKIKIVYRWGQRISFIYDEGGGARGYGRVSEKDA
PKELLQMLEKQGSEQKLISEEDL
(arbitrarily labeled clone B)
(SEQ ID NO: 398)
ATVKFTYQGEEKQVDISKIKHVRRWGQWIWFIYDEGGGAKGWGGVSEKDA
PKELLQMLEKQGSEQKLISEEDL
(arbitrarily labeled clone E)
(SEQ ID NO: 399)
ATVKFTYQGEEKQVDISKIKYVRRWGQYIWFGYDEGGGARGYGYVSEKDA
PKELLQMLEKQGSEQKLISEEDL
(arbitrarily labeled clone F)
(SEQ ID NO: 400)
ATVKFTYQGEEKQVDISKIKRVYRYGQWIWFRYDEGGGAYGGGWVSEKDA
PKELLQMLEKQGSEQKLISEEDL
(arbitrarily labeled clone H)
(SEQ ID NO: 401)
ATVKFTYQGEEKQVDISKIKSVSRWGQAIIFRYDEGGGAKGKGSVSEKDA
PKELLQMLEKARIRTKAYF
(arbitrarily labeled clone K).

Notably, despite the small differences between the compounds in FIGS. 2A-2C, it was found that all of these variants preferentially bound the TF antigen versus the other compounds and the control. Thus, these data illustrate that proteins can be engineered to preferentially bind to specific sugars. Additionally, the variants differed from each other by 6 or fewer amino acids within the binding region.

FIG. 2E shows the biolayer interferometry traces for clone E. Clone E was immobilized on a Ni-NTA tip and dipped into a 1 uM solution of the sugar of interest. The traces show an increase in nm as sugar starts binding to protein on the tip, then a decrease in nm as the tip is moved from the sugar solution to buffer only. From this data, a curved was fitted and the binding affinity was determined from the rate of association and dissociation. FIG. 2E demonstrates that clone E bound to the TF antigen but did not bind to the negative control (PAA) and the other related disaccharides provided.

### Example 4

This example describes certain glycan-binding proteins from Example 1. Some of the variants that were generated in Example 1 demonstrated high specificity for a target of interest and could distinguish small points of differences between molecules that were targeted and other, non-target molecules having similar structures. For instance, in this example, variants were evolved to bind sialic acid (Neu5Ac), as discussed in Example 1. These variants were then studied with flow cytometry and in particular, were determined to preferentially bind to Neu5Ac (sialic acid) relative to Neu5Gc. These variants (from Sequence List 1) have the following sequences:

(SEQ ID NO: 402)
ATVKFTYRGEEKQVGISRIKSVRRIGQWIKFWYDEGSGAYGRGYVSEKDA
PKELLQMLEKR (arbitrarily labeled clone A4)
(SEQ ID NO: 403)
ATVKFTYRGEEKQVGISRIKSVRRIGQWIKFWYDEGSGAYGRGYVSGKDA
PKELLQMLEKR (arbitrarily labeled clone B5)
(SEQ ID NO: 404)
ATVKFTYRGGEKQVGISRIKSVRRIGQWIKFWYDEGSGAYGRGYVSEKDA
PKELLQMLEKR (arbitrarily labeled clone B6)
(SEQ ID NO: 405)
ATVKFTYRGEEKQVGISRIKSVHRIGRWIKFWYDEGSGAYGRGYVSEKDA
PKELLQMLEKR (arbitrarily labeled clone B8)
(SEQ ID NO: 406)
ATVKFTYRGEEKQVGISRIKSVHRIGQWIKFWYDEGSGAYGRGYVSKKDA
PKELLQMLEKR (arbitrarily labeled clone C11)

To analyze this specificity, yeast cells bearing the HA-epitope tag and displaying Sso7d variant Clone B5, for example, on their surface were labeled using fluorescent anti-HA antibody. These were provided 500 nM of the desired sugar-PAA-FITC for 1 hour, then analyzed by analytical flow cytometry for co-localization of both fluorophores, indicating glycan binding. Specific binding can be observed by the percentage of cells binding Neu5Ac versus Neu5Gc or PAA-FITC alone. Cells were gated in flow cytometry parameters to ensure single-cell analysis of live cells presenting Sso7d on their surface. As an example, the binding constant for Clone B5, as determined independently by BLI with soluble, expressed Clone B5, was 25-30 nM.

FIGS. 3A-3B show the structures of sialic acid (Neu5Ac) (FIG. 3A) and Neu5Gc (FIG. 3B). These binding determinants differ by one hydroxyl group.

FIGS. 3C-3E show the flow cytometry results for Neu5Ac-PAA-FITC (FIG. 3C), Neu5Gc-PAA-FITC (FIG. 3D), and the control PAA-FITC (FIG. 3E) for clone B5. These results show that the variants tested preferentially bound to sialic acid relative to Neu5Gc-PAA-FITC or PAA-FITC. Similar results have been attained for other glycan-binding proteins from Example 1, such as clones A4, B6, B8, and C11.

### Example 5

This example describes testing of glycan-binding proteins described herein against various glycans in flow cytometry binding studies.

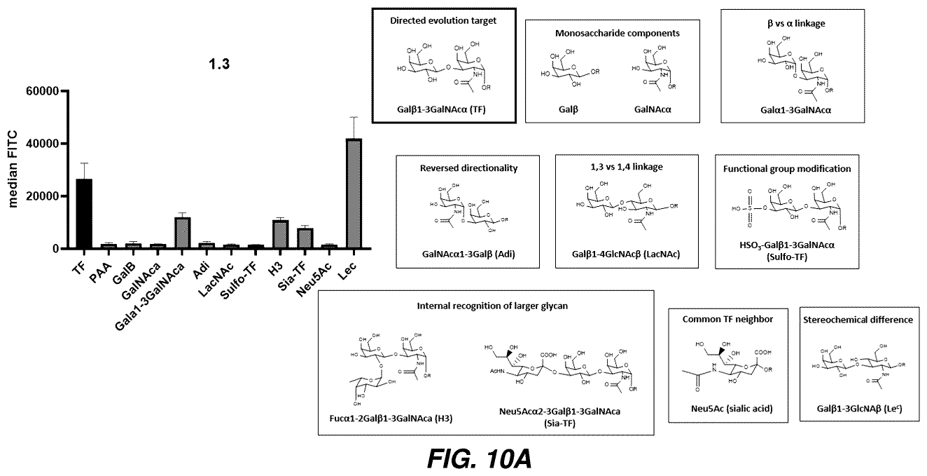

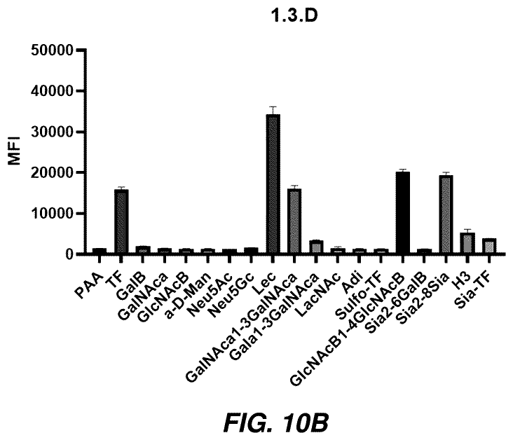

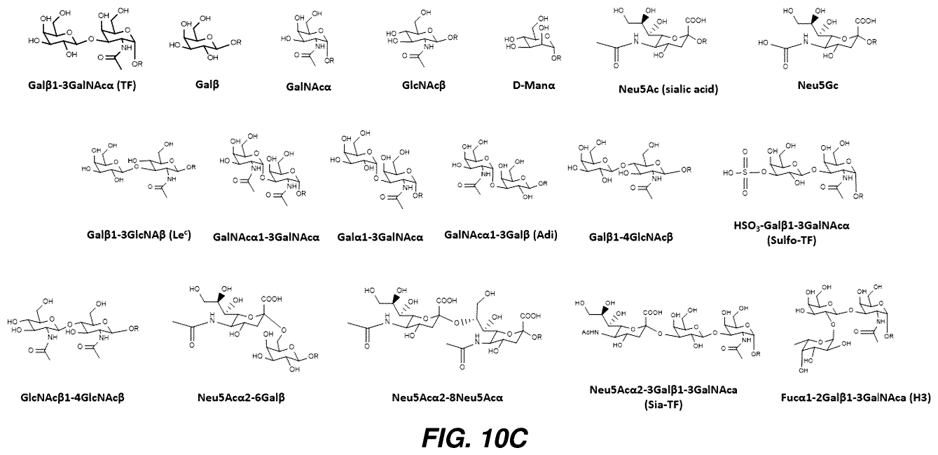

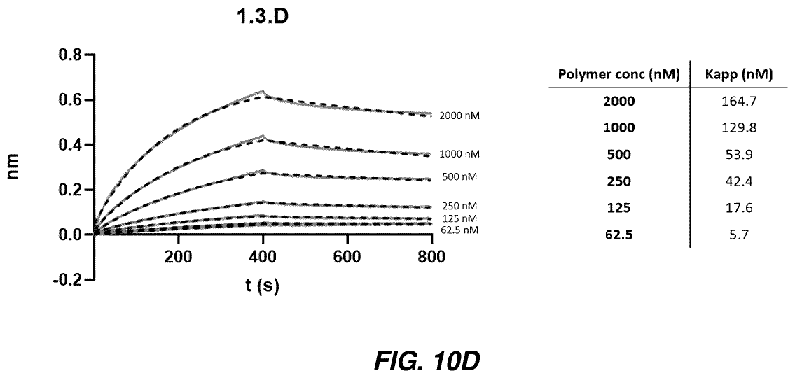

A mixed library of clones was generated based upon the directed evolution target GalP1-3GalNAcα (TF). Based upon the directed evolution target GalP1-3GalNAcα (TF), glycans with structural variations were chosen for a flow cytometry study in which binding behavior was examined (FIG. 10C). Glycans were chosen that possess atom-level differences to each other, including but not limited to: glycans that differ by 1 inverted stereocenter (e.g., GlcNAc vs. GalNAc), glycans with sidechains on neighboring carbon atoms (e.g., OH— on C3 vs. OH— and C4), disaccharides that are comprised of identical monosaccharides whose positions have been flipped (e.g., Gal-GalNAc vs. GalNac-Gal) and others. These glycans with structural variations (FIG. 10C) were all chosen to highlight the ability of this scaffold at distinguishing small structural differences essential to glycan recognition in nature. These results show that only glycan Galβ1-3GlcNAβ (Lec) demonstrated greater binding than the directed evolution target Galβ1-3GalNAcα for a mixed library of clones (FIG. 10A). In addition to the previously discussed binding study, a flow cytometry study in which the binding specificity was studied was carried out, and results show that glycans Galβ1-3GlcNAβ (Lec), GlcNAcβ 1-4GlcNAcβ, and Sia2-8Sia had higher binding specificities than that of TF, while GalNAcα1-3GalNAcα had comparable binding specificity to TF (FIG. 10B). Biolayer interferometry was also used to calculate apparent K_{D} values at varying polymer concentrations for Clone 1.3.D (FIG. 10D), which has the following sequence:

(SEQ ID NO: 416)
ATVKFTYRGEEKQVDISKIKYVRRWGQYIWFGYDEGGGARGYGY
VSETDAPELLLQMLEKQ (Clone 1.3.D).

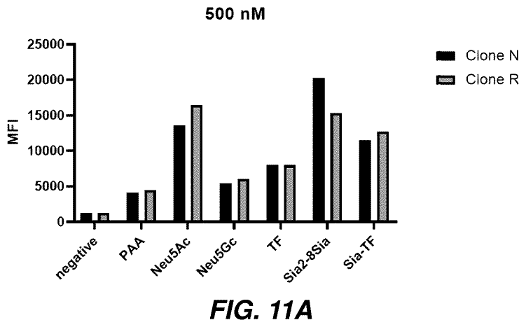

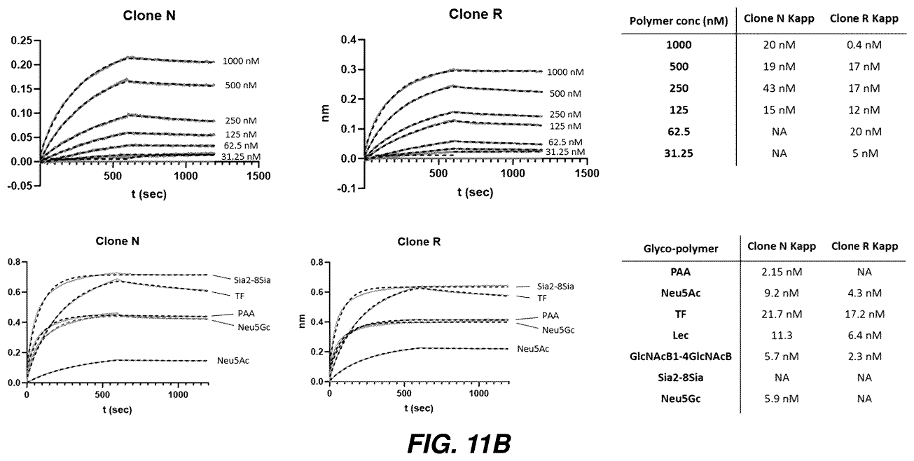

Binding specificity was also tested for various glycans with Clone N and Clone R (FIG. 11A), which have the following sequences:

(SEQ ID NO: 417)
ATVKFTYRGEGKqVGISRIKSVRRIGQWIKFWYDEGSGAYGRGY
VSGKDAPKELLQMLEKR (Clone N)
(SEQ ID NO: 418)
ATVKFTYRGEEKQVGISRIKSVRRIGRWIKLWYDEGSGAYGRGY
VSGKDAPKELLQMLEKR (Clone R)

The results indicate that glycan Sia2-8Sia showed the most difference in preferential binding, as evidenced by the median fluorescence intensity values for Clone N and Clone R binding. Biolayer interferometry was used to calculate K_{D} values for Clone N and Clone R at varying polymer concentrations and using various glycol-polymers (FIG. 11B). These biolayer inferometry results measure average apparent K_{D} values for Clone N and Clone R to be 24 nM and 12 nM respectively, suggesting these scaffolds bind glycans 10- to 100-fold more tightly than glycan-binding proteins occurring in nature (i.e. lectins and mAbs).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above. In some cases, the methods may also have intervening steps in addition to those described.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1-75. (canceled)

76. A method of producing a glycan-binding protein, comprising:
providing a protein scaffold, wherein the protein scaffold has no more than 200 amino acid residues, with a binding face area of less than or equal to 6 square nanometers (nm²);
generating one or more variants of the protein scaffold;
determining binding and/or binding selectivity of the one or more variants to a monosaccharide or disaccharide-binding determinant;
selecting a variant exhibiting increased binding and/or binding selectivity to the monosaccharide or disaccharide-binding determinant from the one or more variants; and
repeating the generating, determining and selecting steps, using the variant exhibiting increased binding and/or binding selectivity to the monosaccharide or disaccharide-binding determinant in each repeat.

77. A method of producing a glycan-binding protein, comprising:
providing a protein scaffold, wherein the protein scaffold has no more than 200 amino acid residues and a melting temperature of greater than or equal to 50° C.;
generating one or more variants of the protein scaffold;
determining binding and/or binding selectivity of the one or more variants to a monosaccharide or disaccharide-binding determinant;
selecting a variant exhibiting increased binding and/or binding selectivity to the monosaccharide or disaccharide-binding determinant from the one or more variants; and
repeating the generating, determining and selecting steps, using the variant exhibiting increased binding and/or binding selectivity to the monosaccharide or disaccharide-binding determinant in each repeat.

78. A method of producing a glycan-binding protein, comprising:
providing a protein scaffold, wherein the protein scaffold has no more than 200 amino acid residues and is devoid of disulfides;
generating one or more variants of the protein scaffold;
determining binding and/or binding selectivity of the one or more variants to a monosaccharide or disaccharide-binding determinant;
selecting a variant exhibiting increased binding and/or binding selectivity to the monosaccharide or disaccharide-binding determinant from the one or more variants; and
repeating the generating, determining and selecting steps, using the variant exhibiting increased binding and/or binding selectivity to the monosaccharide or disaccharide-binding determinant in each repeat.

79. The method of claim 77, wherein the protein scaffold has a melting temperature of greater than or equal to 60° C.

80. The method of claim 77, wherein the protein scaffold has a melting temperature of greater than or equal to 70° C.

81. The method of claim 77, wherein the protein scaffold is devoid of sulfides.

82. The method of claim 77, wherein the protein scaffold has a binding face area of less than or equal to 6 square nanometers (nm²).

83. The method of claim 77, wherein the protein scaffold has a binding face area of at least 4 square nanometers (nm²).

84. The method of claim 77, wherein the protein scaffold has a binding face area of at least 5 square nanometers (nm²).

85. The method of claim 77, wherein the repeating step is repeated at least 5 times.

86. The method of claim 77, wherein generating one or more variants of the protein scaffold comprises generating one or more variants of the protein scaffold that have, on average, greater than or equal to 1 amino acid mutation.

87. The method of claim 77, further comprising producing a variant exhibiting a K_{D} of less than 10⁻⁵ M to the monosaccharide or disaccharide-binding determinant.

88. The method of claim 77, wherein the protein scaffold is compatible with yeast surface display.

89. The method of claim 77, wherein the protein scaffold is synthesized and/or expressed in E. coli.

90. The method of claim 77, wherein the protein scaffold is bioconjugated to a fluorophore, purification tag, biocompatible resin, and/or 2-dimensional (2D) array.

91. The method of claim 77, wherein the protein scaffold has greater than or equal to 50 amino acid residues.

92. The method of claim 77, wherein the protein scaffold has greater than or equal to 75 amino acid residues.

93. The method of claim 92, wherein the protein scaffold has a melting temperature of greater than or equal to 60° C. and less than or equal to 90° C.

94. The method of claim 93, wherein the protein scaffold has a binding face area of at least 5 square nanometers (nm²) and less than or equal to 6 square nanometers (nm²).

95. The method of claim 94, wherein the protein scaffold is devoid of sulfides, is compatible with yeast surface display, is synthesized and/or expressed in E. coli, and is bioconjugated to a fluorophore, purification tag, biocompatible resin, and/or 2-dimensional (2D) array.
